(19) United States
(12) Patent Application Publication
(10) Pub. No.: US 2024/0374136 A1
(43) Pub. Date: Nov. 14, 2024

(54) SYSTEM AND METHOD FOR AUTOMATIC DETECTION OF CLINICAL DETERIORATION EVENTS

(71) Applicants: Danmarks Tekniske Universitet, Kongens Lyngby (DK); Rigshospitalet, Copenhagen Ø (DK); Bispebjerg Hospital, Copenhagen NV (DK)

(72) Inventors: Helge Bjarup Dissing Sørensen, Kongens Lyngby (DK); Eske K. Aasvang, Copenhagen Ø (DK); Christian S. Meyhoff, Copenhagen NV (DK)

(21) Appl. No.: 18/577,921

(22) PCT Filed: Jul. 11, 2022
(86) PCT No.: PCT/EP2022/069262

§ 371 Date: Jan. 9, 2024

(30) Foreign Application Priority Data
Jul. 9, 2021 (EP) 21184712.4
Oct. 29, 2021 (EP) 21205557.8

(51) Int. Cl. A61B 5/00; A61B 5/0205; A61B 5/021; A61B 5/08; A61B 5/145; A61B 5/363

(52) U.S. Cl. CPC A61B 5/0015; A61B 5/0205; A61B 5/021; A61B 5/0816; A61B 5/0826; A61B 5/14542; A61B 5/363

(57) ABSTRACT

Disclosed are a clinical support system and associated method for automatic real-time detection of clinical deterioration events in a patient. Existing clinical support systems typically rely on alarm generation based on simple threshold values. This often results in too many alarms, including false alarms, which consequently results in alarm fatigue in the medical staff or in the ignoring of the alarms. The presently disclosed system and method provides an alternative to existing clinical support systems, since it incorporates clinically validated computer-implemented subroutines that provides a higher predictive value to the medical staff. The subroutines utilize thresholds and time durations, which have been clinically evaluated such that false alarms are reduced while keeping the most relevant alarms, i.e. alarms that require clinical action. Further disclosed is a computer program configured to execute the disclosed method, thereby providing automatic real-time detection of clinical deterioration events in a patient.

The present disclosure relates to a computer-implemented method configured for automatic real-time detection of clinical deterioration events in a patient. The disclosure further relates to a system for carrying out the disclosed method.

### BACKGROUND OF INVENTION

Clinical practice relies on manual recording of physiological patient data every 12 hours to manually calculate an Early Warning Score (EWS) or similar “risk-scores” for detection of patients at need. Selected physiological patient data (blood pressure, respiratory rate, heart/pulse rate, and body temperature) are traditionally described as vital signs. The term vital sign is, however, historically based on those biomarkers for physical status that have been possible to measure (starting with pulse rate and temperature centuries ago). Because peripheral monitoring of blood-pressure and oxygen saturation became reliable and commercially available, this is commonly also included as vital signs. Some hospitals also include parameters such as mental status, pain, urine output, blood glucose and end-tidal CO2 as vital signs and in the EWS. A problem with the EWS approach and similar “track-and-trigger” systems, is that a number of critical events occur between the fixed measurements without being detected. The EWS has a mandatory requirement for more frequent registration depending on the severity of the latest measured values, but EWS has not proved any effect on complications or survival despite intense resource allocation. For example, it has been shown that manual routine EWS measurements detects only 5% of the serious cases of severe oxygen desaturation, when compared to continuous 24/7 automatic measurements. In layman's terms, the main cause for low and late detection rate despite large resource allocation is two-fold: Manual and infrequent collection of patient data is insufficient to detect clinical deterioration prior to the event. Manual, clinical interpretation of threshold values for individual parameters is not sensitive or sophisticated enough to capture events in a timely manner. Trends and combination of physiological parameters of vital signs are too complex to be manually assessed, and vulnerable to the experience or alertness of the individual assessor (e.g. nurse, physician). Nowadays, it is furthermore possible to measure a variety of other biomarkers for physical status, including advanced analyses of heart rate and heart rhythm and peripheral perfusion to describe circulatory function. It is therefore very plausible that new biomarkers for physical status will be added to the traditional list of vital signs introduced above. In the sections below, the term ‘vital signs’ will refer to this indefinite list of biomarkers for the assessment of the physical status of a patient.

In general, clinical decision support systems (CDSS) are focused on using knowledge management in such a way to achieve clinical advice for patient care based on multiple claims of patient data. A problem encountered within many clinical support systems is that the alarm generations are based on simple threshold alerts, which consequently results in too many alarms, many of them being false alarms, whereby the medical staff (nurses, doctors, etc.) is exhausted. This phenomenon is also known as alarm fatigue, which is a well-known and hugely recognized challenge related to monitoring of patients, where alarms will typically be either muted, or threshold values disregarded—or simply ignored.

Therefore, there is a need of a system and method that provides an alternative to simple threshold monitoring for predicting adverse patient events. Specifically, there is a need of a clinical decision support system, wherein the alarm generation is based on intelligent algorithms such that true alarms are maximized and false alarms are reduced, while still providing vital sign alarms, which are clinically actionable.

### SUMMARY OF INVENTION

Currently, the monitoring of post-operative patients relies on intermittent bedside monitoring and simple models of Early Warning Scores (EWS) in the hospital. There is a need of a system that facilitates continuous and predictive monitoring and therefore improves the monitoring of patients.

The present disclosure addresses the above-mentioned challenges by providing a system and method for automatic and continuous detection of clinical deterioration events in a patient. An advantage of the presently disclosed system and method, is that it provides a continuous real-time monitoring of a patient (ideally 24/7), wherein an alarm is generated in case the patient has a deterioration event. The method comprises the step of executing one or more computer-implemented clinically validated automatic deterioration event subroutines, wherein each subroutine is configured to determine a specific clinical deterioration event in the patient.

In particular, the present disclosure relates to a computer-implemented method configured for automatic real-time detection of clinical deterioration events in a patient, the method comprising the steps of:
continuously receiving a plurality of different vital sign data from a plurality of sensors worn by the patient, the vital sign data selected from the group of: electrocardiogram (ECG), photoplethysmogram (PPG), heart rate (HR), respiration rate (RR), blood pressure (e.g. systolic blood pressure, SBP), heart rhythm, ischemic electrocardiographic response, peripheral temperature, peripheral skin conductance, 3D body position and acceleration, pulse rate, peripheral perfusion index, peripheral oxygen saturation (SpO₂), and subcutaneous glucose concentration; optionally/alternatively vital sign data like blood pressure can be estimated from other measured vital sign data, for example heart rate (HR), respiration rate (RR), blood oxygen saturation (SpO2) and pulse rate (PR) as disclosed herein, as these vital sign data are easier to measure than blood pressure,
analyzing the vital sign data to identify artefacts;
discarding one or more data samples associated with the identified artefacts in the vital sign data in order to continuously obtain validated patient vital sign parameters;
executing one or more computer-implemented clinically validated deterioration event subroutines, each subroutine configured to receive one or more of the validated vital sign parameters and determine a specific clinical deterioration event in the patient, said clinical deterioration event selected from the group of:
   bradypnea/apnea based on validated heart rate and respiration rate parameters,
   tachypnea based on validated respiration rate,
   hypoventilation based on validated respiration rate and peripheral arterial oxygen saturation,
   desaturation based on validated peripheral arterial oxygen saturation,
   sinus tachycardia based on validated heart rate,
   bradycardia based on validated heart rate,
   hypotension based on validated systolic blood pressure, or estimated systolic blood pressure
   circulatory collapse based on validated heart rate and validated systolic blood pressure or estimated systolic blood pressure,
   asystole based on validated heart rhythm and pulse rate,
   hypertension based on validated systolic blood pressure or estimated systolic blood pressure,
   atrial fibrillation based on validated heart rhythm,
   ventricular extrasystoles based on validated heart rhythm, and
   ventricular tachycardia/-fibrillation based on validated heart rhythm,
providing an alarm when at least one of said deterioration events has been detected by one of said clinically validated automatic deterioration event subroutines.

Optionally and/or alternatively the one or more computer-implemented clinically validated deterioration event subroutines can be executed based on forecasted vital signs, as for example shown in example 5, where the vital sign parameters heart rate and respiration rate are forecasted based on modelling, e.g. machine learning, in particular Multivariate Auto-Regressive (MAR) models, of the validated vital sign parameters HR and RR. Any vital sign parameters can be forecasted based on this approach, i.e. forecasted for at least 5, 10, 15, 30, 45 or even 60 minutes. Each subroutine based on forecasted vital signs can then be configured to receive one or more of the forecasted vital sign parameters and determine a specific forecasted clinical deterioration event in the patient, said clinical deterioration event selected from the group of clinical deterioration events listed above. In that way alarms can be generated based on forecasted data, i.e. before the deterioration event actually happens and/or predicting whether the deterioration event is likely to occur in the near future.

Preferably, all vital signs are received continuously. The data may be transmitted with different sampling frequencies and it may be transmitted blockwise (sampling). As also described in Example 3 herein blood pressure (both systolic and diastolic) can be estimated on other measured vital signs, for example HR, RR, SpO₂ and PR, such that blood pressure can be measured both cuffless and non-invasively, and possibly be validated because it can be based on validated vital signs data, and thereby possibly replace the validated systolic blood pressure measurement used in the subroutines as described herein.

Each clinically validated deterioration event subroutine is associated with one or more criterions, which determine whether an alarm should be generated. The criterions comprise thresholds and time duration(s), which have been clinically determined and evaluated by medical doctors, such that the amount of alarms generated are reduced and more important alarms are generated. Hence, the presently disclosed system and method provides much more predictive value than existing systems, since it provides alarms of events that require clinical action from the medical staff, while simultaneously greatly reducing the amount of false alarms. This has been achieved by engineering a plurality of deterioration event subroutines, also referred to as predictive computer algorithms.

The present disclosure further relates to a system for automatic detection of a clinical deterioration event in a patient, said system comprising:
one or more sensors configured for automatically monitoring vital sign data (such as heart rate, respiration rate, heart rate variability, temperature, oxygen saturation and blood pressure) of the patient, the one or more sensors further configured for transmitting the vital sign data wirelessly to a server and/or to a gateway;
a first server for receiving and storing the vital sign data, the first server having a computer program thereon, said computer program configured for executing the presently disclosed method thereby providing automatic detection of a clinical deterioration event in a patient; and
one or more gateways configured to provide a wireless communication link between the sensor(s) and the first server.

The system described herein is configured for executing the presently disclosed method thereby providing automatic detection of a clinical deterioration event in a patient. The disclosed system and its functionality is shown in FIGS. 1-3 and further described in the detailed description of the invention.

The disclosure further relates to a computer program having instructions thereon which, when executed by a computing device or system, causes the computing device or system to execute the method disclosed herein, thereby providing automatic real-time detection of clinical deterioration events in a patient.

Accordingly, the presently disclosed system and method provides continuous 24/7 monitoring of patients, wherein clinical deterioration events in the patient is automatically detected and reported through intelligent alarm generation. Specifically, this is achieved by executing a plurality of deterioration event subroutines, which receives input from one or more sensors associated with the patient, wherein said subroutines are configured to provide an alarm in case of a clinical deterioration event in the patient.

Accordingly, the presently disclosed system and method provides a significant improvement to existing clinical support systems, which typically rely heavily on simple thresholds for generating alarms.

The present disclosure further relates to a system for identifying unauthorized access of an account of an online service, comprising a non-transitive, computer-readable storage device for storing instructions that, when executed by a processor, performs a method for identifying unauthorized access of an account of an online service according to the described method. The system may comprise a mobile device comprising a processor and a memory and being adapted to perform the method but it can also by a stationary system or a system operating from a centralized location, and/or a remote system, involving e.g. cloud computing. The invention further relates to a computer program having instructions which when executed by a computing device or system cause the computing device or system to identify an unauthorized access of an account of an online service according to the described method. Computer program in this context shall be construed broadly and include e.g. programs to be run on a PC or software designed to run on smartphones, tablet computers or other mobile devices. Computer programs and mobile applications include software that is free and software that has to be bought, and also include software that is distributed over distribution software platforms such as Apple App Store, Google Play and Windows Phone Store.

### DESCRIPTION OF DRAWINGS

FIG. 1 shows an embodiment of a system for automatic detection of a clinical deterioration event in a patient according to the present disclosure.

FIG. 2 shows a block diagram, which illustrates the overall functionality of the system and method as disclosed herein.

FIG. 3 shows another representation of an embodiment of a system according to the present disclosure.

FIG. 4 shows an embodiment of the ECG preprocessing subroutine according to the present disclosure.

FIG. 5 shows an embodiment of the SpO₂ preprocessing subroutine configured to assess the quality of the SpO₂ values from a pulse oximeter worn by the patient.

FIG. 6 shows a block diagram of the bradypnea subroutine according to one embodiment.

FIG. 7 shows a block diagram of the tachypnea subroutine according to one embodiment.

FIG. 8 shows a block diagram of the hypoventilation subroutine according to one embodiment.

FIG. 9 shows a block diagram of the desaturation subroutine according to one embodiment.

FIG. 10 shows a block diagram of the desaturation subroutine according to one embodiment.

FIG. 11 shows a block diagram of the desaturation subroutine according to one embodiment.

FIG. 12 shows a block diagram of the desaturation subroutine according to one embodiment.

FIG. 13 shows a block diagram of the sinus tachycardia subroutine according to one embodiment.

FIG. 14 shows a block diagram of the sinus tachycardia subroutine according to one embodiment.

FIG. 15 shows a block diagram of the bradycardia subroutine according to one embodiment.

FIG. 16 shows a block diagram of the bradycardia subroutine according to one embodiment.

FIG. 17 shows a block diagram of the hypotension subroutine according to one embodiment.

FIG. 18 shows a block diagram of the hypertension subroutine according to one embodiment.

FIG. 19 shows a block diagram of the hypotension subroutine according to one embodiment.

FIG. 20 shows a block diagram of the hypertension subroutine according to one embodiment.

FIG. 21 shows a block diagram of the circulatory collapse subroutine according to one embodiment.

FIG. 22 shows a block diagram of the atrial fibrillation subroutine according to one embodiment.

FIG. 23 shows a block diagram, which illustrates the overall functionality of the presently disclosed system and method.

FIG. 24 shows a block diagram, which illustrates how the electrocardiogram (ECG) data and photoplethysmogram (PPG) data is handled.

FIG. 25 shows a diagram of the deep generative model (DGM) used in example 1.

FIG. 26 shows a diagram of (a) the inference model and (b) the generative model of the proposed network used in example 1.

FIG. 28 shows the input segment and corresponding reconstruction of chosen samples used in example 1.

FIG. 29 shows the distribution of the samples for the test set in the latent space used in example 1.

FIG. 30 shows depicts the steps of the algorithm used in example 2.

FIG. 31A shows the relation between prediction window and overlap window used in example 2.

FIG. 31B illustrates the extraction of control samples as used in example 2.

FIG. 32 shows the patient inclusion process applied in example 4.

FIGS. 33-34 are visualizations of the night extraction process. In FIG. 33 the patient has an SAE on day 2 (red line), therefore the night before (in green) gets selected. The others night (in gray) are discarded. FIG. 34 shows a patient without SAEs, therefore all nights get included (in cyan).

FIG. 35 shows time series vital signs for the patients used to fit the MAR model used in example 5.

FIG. 36 shows a probabilistic graphical model of the implemented pooled MAR model used in example 5.

FIG. 37 illustrates the setup used for evaluating the model in example 5 on new patients. For each step a forecast (right box) is performed based on the data available in the model window (left box). The windows are then moved 10 minutes forward and the process is repeated.

FIG. 38 shows a visualization of the response of the hierarchical AR-model fitted to the HR and RR data as used in example 5.

### DETAILED DESCRIPTION OF THE INVENTION

The present disclosure relates to a computer-implemented method configured for automatic real-time detection of clinical deterioration events in a patient.

### Vital Sign Data

In a preferred embodiment, the first step of the method is receiving a plurality of different vital sign data from a plurality of sensors worn by the patient. The vital sign data may be selected from the group of: electrocardiogram (ECG), photoplethysmogram (PPG), heart rate (HR), respiration rate (RR), blood pressure (e.g. systolic blood pressure, SBP), heart rhythm, ischemic electrocardiographic response, peripheral temperature, peripheral skin conductance, 3D body position and acceleration, pulse rate (PR), peripheral perfusion index, peripheral oxygen saturation (SpO₂) (e.g. derived from PPG), and subcutaneous glucose concentration. The vital sign data may be received continuously or it may be received at predefined time intervals, such as every minute. The vital signs may have fixed sampling frequencies and some may have block-wise sampling. As an example, ECG may be delivered every first 10 seconds per minute i.e. the next package of 10 seconds of samples are then sent after 50 seconds and so on.

### Removal of Artefacts

In a preferred embodiment, the next step of the method is analyzing the vital sign data to identify artefacts in the data. Artefacts and noise are preferably taken care of for each vital sign whenever needed. Artefacts should be understood as erroneous data displaying unphysical values arising from external factors, which influences the measurement (of the sensors) such that the measurement is disturbed or altered from its true value. An example is motion-related artefacts, e.g. if the patient makes sudden movements, this may influence some of the measured vital sign data. Another example is if one of the sensors is placed incorrectly, it may be unable to measure the intended vital sign. As an example, if the pulse oximeter measures a negative value or more than 100% SpO₂, such data points will be considered artefacts and consequently removed. Therefore, a next step of the method is preferably to discard one or more data samples (i.e. sets of data points) associated with the identified artefacts in the vital sign data in order to obtain validated patient vital sign parameters. Artefacts and noise are estimated by the overall approach to look for abnormal deviation as a function of time, amplitude and frequency content.

### ECG Preprocessing

In a preferred embodiment, the method further comprises the step of executing an ECG preprocessing subroutine configured to assess the quality of the ECG data from an ECG sensor worn by the patient. Some vital sign data, such as RR interval (RRI), PP interval (PPI), HR, heart rhythm and RR are estimated/calculated based on R peak detection in the ECG data, i.e. validated heart rate and validated heart rhythm are typically based on ECG data. The RR interval (RRI) and PP interval (PPI) represent cardiac beat-to-beat interval extracted from ECG and PPG signals, respectively. R peak is understood to have its common meaning, i.e. the maximum amplitude in the R wave in the QRS complex of an electrocardiogram. The system associated with the disclosed method is preferably configured to provide such vital sign data automatically, i.e. automatically perform the R peak detection in the ECG data. However, if the ECG data is noisy or distorted, the detection of the R peak(s) can be faulty, leading to erroneous estimations of RRI, HR, and RR. Therefore, the system is preferably further configured to stream parts of ECG data. The purpose of the ECG preprocessing subroutine is to evaluate whether the streamed parts of ECG data (also referred to as ECG samples) have an acceptable quality. The output of the ECG preprocessing subroutine is a plurality of parameters (goodForHR, goodForAF, goodForRR, goodForMorph), which can obtain a value of either 1 or 0. A value of 1 indicates that the ECG sample is good enough to be used for deriving vital sign data (HR, RR, and/or RRI), which can be used as input to the clinically validated deterioration event subroutines. In that case, the vital sign data is referred to as validated vital sign data. Conversely, a value of 0 indicates that the concerned ECG sample and/or derived vital sign data should be discarded and not applied in the deterioration event subroutines. The parameters goodForHR and goodForRR means that the concerned ECG sample is of good enough quality (in case of a value of 1) to be used to estimate the heart rate (HR), the heart rhythm and respiratory rate (RR) of the patient based on the ECG sample, respectively. Similarly, the parameter goodForAF means that the concerned ECG sample is of good enough quality to be used in the atrial fibrillation (AF) subroutine. The parameter goodForMorph indicates that the ECG sample is good enough (in terms of noise, e.g. quantified by signal-to-noise ratio) for calculating other values from the ECG morphology. In one embodiment, the ECG preprocessing subroutine comprises the steps of:
receiving ECG values from an ECG sensor, the ECG values comprising ECG time stamp, ECG samples, heart beat time stamp, R-R interval, and/or QRS amplitude;
discarding ECG values that are outside predefined thresholds, such as ECG<0 or ECG>4000 (representing 12 bit resolution i.e. the number representing realistic heart values), and/or discarding ECG values that are duplicate;
performing a linear interpolation of ECG missing values;
determining QRS index (i.e. the major heart peak sample number within a predefined time window such as a 10 seconds window) in ECG using QRS time stamp;
comparing the number of heart beats and/or the QRS amplitude and/or the ratio between a sub selection of the QRS amplitudes and the number of heartbeats to one or more predefined thresholds, and in case said thresholds are exceeded, discarding one or more of the ECG values;
performing a bandpass filtering of the ECG values;
correcting R peak(s) from the ECG values and/or normalizing each heart beat from the ECG values based on heuristic rules;
calculating ecgTemplate (i.e. an average ECG heart cycle calculated from the first heart cycles in a heart signal) or previously received ecgTemplate;
calculating correlation coefficients (cc);
comparing the average of cc and/or the number of cc to one or more predetermined thresholds, and in case said thresholds are exceeded, discarding one or more of the ECG values;
calculating the deviation between the corrected R peak(s) and the non-corrected R peak(s) and storing said deviation as HBdev value(s); and
comparing the HBdev value(s) to one or more predefined thresholds and discarding the ECG values that are associated with HBdev value(s) that exceed the one or more predefined thresholds.

### SpO₂ Preprocessing

In a preferred embodiment, the method further comprises the step of executing a SpO₂ preprocessing subroutine configured to assess the quality of the SpO₂ data from a pulse oximeter worn by the patient in order to obtain validated SpO₂ data. The system associated with the presently disclosed method is preferably configured to receive SpO₂ data from the pulse oximeter with a given sampling frequency, e.g. 1 Hz. A given time segment (e.g. length of 1 minute) of data comprising a number of SpO₂ values, is represented by an average SpO₂ sample (i.e. one value representing the oxygen level for a one minute interval), may be transferred e.g. every minute, to the one or more servers storing the computer program for executing the disclosed method. In one embodiment, the SpO₂ preprocessing subroutine comprises the steps of:
receiving SpO₂ data (such as SpO₂ data samples) from a pulse oximeter worn by the patient;
removing SpO₂ data values in the SpO₂ data or SpO₂ data samples that exceed one or more predefined thresholds such as SpO₂<0 and/or SpO₂>100;
removing duplicate SpO₂ data;
removing SpO₂ data that has a difference per second greater than 4;
extrapolating missing SpO₂ data; and
optionally calculating the average SpO₂ value from the SpO₂ data.

### Deterioration Event Subroutines

In a preferred embodiment, the next step of the method is executing one or more computer-implemented clinically validated deterioration event subroutines. Preferably, each subroutine is configured to receive one or more of the validated vital sign parameters and determine a specific clinical deterioration event in the patient. The clinical deterioration event may be selected from the group of: bradypnea/apnea, tachypnea, hypoventilation, desaturation, sinus tachycardia, bradycardia, hypotension, circulatory collapse, hypertension, atrial fibrillation, ventricular extrasystoles, ventricular tachycardia/-fibrillation (VT/VF), asystole, cardiac ischemia, low perfusion index, and acute stress. Each deterioration event subroutine is described in further details in the following.

### Bradypnea Subroutine

The disclosed method may comprise a bradypnea subroutine configured to determine bradypnea/apnea. According to one embodiment, the bradypnea subroutine comprises the steps of:
receiving validated HR and RR values;
comparing the validated HR and RR values to one or more predefined bradypnea thresholds, and
providing an alarm in case the HR and RR values exceed said predefined bradypnea thresholds for a predefined time duration.

The one or more bradypnea thresholds may be selected from the group of: HR>10 bpm, HR>15 bpm, HR>20 bpm, HR>25 bpm, RR≤3 bpm, RR≤5 bpm, RR≤10 bpm, RR≤15 bpm, and/or combinations thereof. In a preferred embodiment, the bradypnea subroutine comprises the bradypnea thresholds HR>20 and RR≤5. The predefined time duration may be selected from the group of: ≥1 min, ≥2 min, ≥3 min, ≥5 min, or ≥10 min. In a preferred embodiment, the bradypnea subroutine provides an alarm in case HR>20 and RR≤5 for more than 1 minute.

### Tachypnea Subroutine

The disclosed method may comprise a tachypnea subroutine configured to determine tachypnea. According to one embodiment, the tachypnea subroutine comprises the steps of:
receiving validated RR value(s);
comparing the validated RR value(s) to a predefined tachypnea threshold; and
providing an alarm in case the RR value(s) exceed the tachypnea threshold for a predetermined time duration.

The predefined tachypnea threshold may be selected from the group of: RR≥20 bpm, RR≥24 bpm, RR≥28 bpm, and/or combinations thereof. In a preferred embodiment, the tachypnea subroutine comprises the tachypnea threshold: RR≥24 bpm. The predefined time duration may be selected from the group of: ≥1 min, ≥2 min, ≥3 min, ≥5 min, ≥10 min. A time duration of ≥5 min is preferred. In a preferred embodiment, the tachypnea subroutine provides an alarm in case RR≥24 bpm for more than 5 minutes.

### Hypoventilation Subroutine

The disclosed method may comprise a hypoventilation subroutine configured to determine hypoventilation. According to one embodiment, the hypoventilation subroutine comprises the steps of:
receiving validated RR and SpO₂ value(s);
comparing each of the validated RR and SpO₂ values to one or more hypoventilation thresholds; and
providing an alarm in case in case the RR and SpO₂ values exceed the hypoventilation threshold(s) for a predefined time duration.

The hypoventilation thresholds may be selected from the group of: RR<15 bpm, RR<13 bpm, RR<11 bpm, RR<9 bpm, SpO₂<92%, SpO₂<90%, SpO₂<88%, SpO₂<86%, and/or combinations thereof. In a preferred embodiment, the hypoventilation thresholds comprise RR<11 bpm and SpO₂<88%. The predefined time duration may be selected from the group of: ≥1 min, ≥2 min, ≥3 min, ≥5 min, or ≥10 min. A time duration of ≥5 min is preferred. In a preferred embodiment, the hypoventilation subroutine provides an alarm in case RR<11 bpm and SpO₂<88% for more than 5 minutes.

### Desaturation Subroutine

The disclosed method may comprise a desaturation subroutine configured to determine desaturation. According to one embodiment, the desaturation subroutine comprises the steps of:
receiving validated SpO₂ values;
comparing the validated SpO₂ values to one or more predefined SpO₂ thresholds; and
providing an alarm in case the SpO₂ values exceed the SpO₂ threshold(s) for a predefined time duration t.

The predefined SpO₂ thresholds may comprise any of: SpO₂<92%, SpO₂<88%, SpO₂<85%, SpO₂<80%, and/or combinations thereof. The predefined time duration may be selected from the group of: ≥1 min, ≥5 min, ≥10 min, ≥30 min, or ≥60 min. In a preferred embodiment, the desaturation subroutine provides an alarm in case:

$$SpO 2 < 92 \% for t \geq 60 min ,$$

$$or$$

$$SpO 2 < 88 \% for t \geq 10 min ,$$

$$or$$

$$SpO 2 < 85 \% for t \geq 5 min ,$$

$$or$$

$$SpO 2 < 80 \% for t \geq 1 min .$$

### Sinus Tachycardia

The disclosed method may comprise a sinus tachycardia subroutine configured to determine sinus tachycardia, said subroutine comprising the steps of:
receiving validated HR value(s);
comparing the validated HR values to one or more predefined sinus tachycardia thresholds; and
providing an alarm in case the HR values exceed the sinus tachycardia threshold(s) for a predefined time duration t.

The one or more predefined sinus tachycardia thresholds may be selected from the group of: HR≥100 bpm, HR≥111 bpm, HR≥120 bpm, or HR>130 bpm. The predefined time duration may be selected from the group of: ≥5 min, ≥10 min, ≥30 min, >60 min, or ≥80 min. In a preferred embodiment, the sinus tachycardia subroutine provides an alarm in case: HR>130 bpm for t≥30 min, or in case HR≥ 111 bpm for t≥60 min.

### Bradycardia Subroutine

The disclosed method may comprise a bradycardia subroutine configured to determine bradycardia, said subroutine comprising the steps of:
receiving validated HR value(s);
comparing the validated HR values to one or more predefined bradycardia thresholds and/or ranges; and
providing an alarm in case the HR values exceed the bradycardia threshold(s) and/or ranges for a predefined time duration t.

The bradycardia thresholds/ranges may be selected from the group of: HR<40 bpm, HR<30 bpm, HR<25 bpm, 25 bpm≤ HR≤45 bpm, or 30 bpm≤ HR≤40 bpm. The predefined time duration may be selected from the group of: ≥1 min, ≥2 min, ≥3 min, ≥5 min, or ≥10 min. In a preferred embodiment, the bradycardia subroutine provides an alarm in case HR<30 bpm for t≥1 min, or in case 30 bpm≤ HR≤40 bpm for t≥5 min. Preferably, the alarm is only provided if the parameter goodForHR is equal to 1.

### Hypotension Subroutine

The disclosed method may comprise a hypotension subroutine configured to determine hypotension, said subroutine comprising the steps of:
receiving validated SBP value(s);
comparing the validated SBP values to one or more predefined hypotension thresholds; and
providing an alarm in case the SBP values exceed the hypotension threshold(s) for one or more consecutive measurements and/or for a predefined time duration t.

The hypotension thresholds may be selected from the group of: SBP<91 mmHg, SBP<80 mmHg, SBP<70 mmHg, SBP<60 mmHg, and/or combinations thereof. In a preferred embodiment, the hypotension subroutine provides an alarm in case SBP<91 mmHg for two consecutive measurements, or in case SBP<70 mmHg.

### Circulatory Collapse

The disclosed method may comprise a circulatory collapse subroutine configured to determine circulatory collapse, said subroutine comprising the steps of:
receiving validated SBP and HR value(s);
comparing the validated SBP and HR values to one or more predefined SBP thresholds and HR thresholds; and
providing an alarm in case the SBP threshold(s) and at least one of the HR thresholds is exceeded for a predefined time duration t.

The predefined SBP threshold(s) may be selected from the group of: SBP<110 mmHg, SBP<100 mmHg, and/or SBP<90 mmHg. The predefined HR thresholds may be selected from the group of: HR>110 bpm, HR>120 bpm, HR>130 bpm, HR<60 bpm, HR<50 bpm, HR<40 bpm, and/or combinations thereof. The predefined time duration may be selected from the group of: ≥1 min, ≥5 min, ≥10 min, ≥30 min, or ≥60 min. In a preferred embodiment, the circulatory collapse subroutine provides an alarm in case:

$$SBP < 100 mmHg and HR > 110 bpm for t \geq 30 min ,$$

$$or$$

$$SBP < 100 mmHg and HR > 130 bpm for t \geq 5 min ,$$

$$or$$

$$SBP < 100 mmHg and HR < 50 bpm for t \geq 30 min .$$

### Asystole Subroutine

The disclosed method may comprise an asystole subroutine configured to determine asystole, said subroutine comprising the steps of:
receiving ECG data samples;
detecting QRS-complexes in the ECG data samples;
providing an alarm in case there are no QRS-complexes detected for a predefined time duration t1, and/or in case there is no pulse detected for a predefined time duration t2.

The predefined time durations t1 and t2 may be more than 10 seconds, or more than 15 seconds, or more than 20 seconds, or more than 25 seconds, or more than 30 seconds.

### Hypertension Subroutine

The disclosed method may comprise a hypertension subroutine configured to determine hypertension, said subroutine comprising the steps of:
receiving validated SBP value(s);
comparing the validated SBP values to one or more predefined hypertension thresholds; and
providing an alarm in case the SBP values exceed the hypertension threshold(s) for one or more consecutive measurements and/or for a predefined time duration t.

The hypertension threshold(s) may be selected from the group of: SBP≥180 mmHg, SBP≥190 mmHg, SBP≥200 mmHg, SBP≥210 mmHg, SBP≥220 mmHg, and/or combinations thereof. The predefined time duration may be selected from the group of: ≥1 min, ≥5 min, ≥10 min, ≥30 min, or >60 min. In a preferred embodiment, the hypertension subroutine provides an alarm in case SBP≥180 mmHg for t≥60 min, or in case SBP≥220 mmHg for at least one measurement.

### Atrial Fibrillation Subroutine

The disclosed method may comprise an atrial fibrillation subroutine configured to determine atrial fibrillation, said subroutine comprising the steps of:
receiving validated RRI values and/or RRI samples;
comparing the validated RRI values to one or more predefined RRI threshold(s);
removing RRI values that exceed the predefined RRI threshold(s);
optionally comparing the amount of RRI values (e.g. the length of an array storing RRI values) to a second RRI threshold;
computing the normalized difference (NDR) of the RRI and storing these as NDR values;
optionally removing the NDR values that fall outside predefined percentiles;
providing the NDR values to a supervised learning model, e.g. a Support Vector Machine (SVM) model, trained and configured to determine atrial fibrillation (AF) based on the NDR values; and providing an alarm in case of atrial fibrillation.

The predefined RRI threshold(s) may be selected from the group of: RRI<300, RRI<200, RRI<150, RRI>2500, RRI>3000, or RRI>3500. The second RRI threshold may be that the size of the RRI array storing the RRI values is greater than 15, or greater than 20, or greater than 25, or greater than 30. The atrial fibrillation subroutine preferably comprises the step of computing the normalized difference of the validated RRI values and storing the computed normalized difference values in a stored set of NDR values. The atrial fibrillation subroutine may further comprise the step of removing the NDR values that fall outside predefined percentiles of the values. Said predefined percentiles may comprise the 10^{th} percentile and the 90^{th} percentile, such that NDR values that are below the 10^{th} percentile and/or above the 90^{th} percentile are removed from the stored set of NDR values. The atrial fibrillation subroutine may further comprise the step of providing the stored set of NDR values to a Support Vector Machine (SVM) model configured to determine the presence of atrial fibrillation. In one embodiment, an SVM model was separately trained for binary classification (of atrial fibrillation) using a radial basis function (RBF) kernel. The misclassification costs were set to be proportional to the number of the training samples for each class. The feature NDR was extracted from a plurality of RRI samples, each sample comprising RRI values from a timespan of one minute. These RRI samples were fed into the SVM model for AF detection.

The disclosed method may comprise yet another atrial fibrillation subroutine configured to determine atrial fibrillation, said subroutine comprising the steps of:
receiving ECG data;
optionally executing a ECG preprocessing subroutine to obtain validated ECG data based on the received ECG data, preferably the ECG preprocessing as disclosed herein;
providing the (validated) ECG data to a semi-supervised learning model, e.g. a deep generative model based on neural networks, trained and configured to determine atrial fibrillation (AF) based on (validated) ECG data (as exemplified in example 1); and
providing an alarm in case of atrial fibrillation.

The semi-supervised learning model may have been trained on less than 50% labelled data, preferably less than 40% labelled data, more preferably less than 30% labelled data, even more preferably less than 20% labelled data, most preferably less than 10% labelled data.

### VT/VF Subroutine

A reference subroutine for ventricular fibrillation (VF) detection can be found in Ibtehaz et al., “VFPred: A fusion of signal processing and machine learning techniques in detecting ventricular fibrillation from ECG signals”, Biomedical Signal Processing and Control 49 (2019), pp. 349-359. The VFPred algorithm can detect VF, which contains the classes VF and non-VF, and can be expanded with the classes VT/VF and non-VT/VF such that both VF and ventricular tachycardia (VT) can be detected.

### Serious Adverse Events

Conventional bedside monitoring system has demonstrated the difficulty in long term monitoring of post-operative patients because the majority of them are ambulatory. With the presently disclosed approach employing wearable sensors and advanced data analytics, those patients will benefit greatly from continuous and predictive monitoring. A Serious Adverse Event (SAE) is any untoward medical occurrence or effect at any dose, any undesirable or unintentional effect that:
results in death (regardless of cause)
is life threatening
results in hospitalization or prolongation of existing hospitalization,
results in persistent or significant disability or incapacity of the subject
is associated with a congenital anomaly or birth defect
is qualified as “other” important medically significant event or condition e.g. the event may jeopardize the subject or may require intervention to prevent one of the outcome listed above (e.g. intensive treatment in an emergency room or at home).

Examples of SAEs are Pneumonia, wound infection. anastomosis leakage, pneumothorax, bleeding, myocardial infarction, pulmonary embolism, delirium, syncope, stroke, transient ischaemic attack, respiratory failure, atelectasis, pneumothorax, pleural effusion, pulmonary embolism, heart failure, deep vein thrombosis, non-fatal cardiac arrest, troponin elevation, myocardial infarction, atrial fibrillation, atrial flutter, ventricular tachycardia, other supraventricular tachyarrhytmias, second-degree atrio-ventricular block, third-degree atrio-ventricular block, urinary tract infection, sepsis, septic shock, surgical site infection, major bleeding, drain, acute renal failure, hypoglycemia, diabetic ketoacidosis, intestinal obstruction, fracture, opiod intoxication, re-operation, and death.

SAEs like atrial fibrillation, atrial flutter, ventricular tachycardia, other supraventricular tachyarrhytmias, second-degree atrio-ventricular block, and third-degree atrio-ventricular block are examples deterioration events that both can be termed clinical deterioration events, and thereby be detected according to the presently disclosed approach, and be termed SAE because they also fall within the definition of SAE as stated above.

Example 2 discloses detection of serious adverse events (SAE) based on machine learning, where a support vector machine model has been trained on validated data. The feature input to the model was extracted from time series of four vital sign parameters HR, RR, SpO2 and sysBP, from where clinical deterioration events were extracted as trends in the data time series. However, the model could equally well have been trained based on features selected from one or more of the specific clinical deterioration events disclosed herein. I.e. once the model is trained as described in example 2, the input to the prediction of SAE will be vital sign data and detection of one or more clinical deterioration events as disclosed herein.

Application of the approach disclosed in example also 2 applies to clinical deterioration events detected in accordance with the presently disclosed approach, i.e. clinical deterioration events and/or SAEs can be detected, and thereby also possibly predicted and preferably prevented, with detection of clinical deterioration events as disclosed herein, i.e. by application of machine learning and continuous vital sign monitoring of (post-operative) patients.

### Nighttime Monitoring

As disclosed in example 4 nighttime monitoring of patient can improve prediction of SAE's, in particular patients having an increased heart rate and breathing rate as well as a slightly lower oxygen saturation during sleep during the nighttime, e.g. from midnight to 6 AM, compared to their normal vital sign parameters, have an increased risk of developing a SAE during the following day. This can be improved by combining the monitoring with a sleep stage detector, for example based on EEG measurements, such that it is known when the patient sleeps such that only sleep vital sign data is used in the nighttime analysis. The observation of an abnormal nighttime period of a patient may trigger an alarm, or a pre-alarm, such that the patient is surveyed more closely the following day and/or by adjusting one or more of the subroutine thresholds such that an alarm is generated earlier.

### Alarm Generation

The presently disclosed method is configured for providing an alarm when at least one deterioration event has been detected by one of the described deterioration event subroutines. Each subroutine receives one or more validated vital sign parameters (such as RR, HR, SpO₂, and SBP) and provides an alarm in case the monitored parameter(s) exceed one or more predefined thresholds for a predefined time duration as explained in further detail in relation to each subroutine. The preferred values of the different thresholds and durations for alarm generation associated with the different subroutines are summarized in the table below.

| Subroutine | Threshold | | Duration | |
|---|---|---|---|---|
| Bradypnea | | RR ≤ 5 bpm and HR > 20 bpm | | ≥1 min |
| Tachypnea | | RR ≥ 24 bpm | | ≥5 min |
| Hypoventilation | | RR < 11 bpm and SpO₂ < 88% | | ≥5 min |
| Desaturation | 1. | SpO₂ < 80% | 1. | ≥1 min |
| | 2. | SpO₂ < 85% | 2. | ≥5 min |
| | 3. | SpO₂ < 88% | 3. | ≥10 min |
| | 4. | SpO₂ < 92% | 4. | ≥60 min |
| | 5. | (optional) >10% reduction from a | 5. | ≥60 min |
| | | dynamic set point, which in example can | | |
| | | be defined as a rolling average of SpO2 | | |
| | | values for ≥6 hours. | | |
| Sinus | 1. | HR > 130 bpm | 1. | ≥30 min |
| tachycardia | 2. | HR ≥ 111 bpm | 2. | ≥60 min |
| Bradycardia | 1. | HR < 30 bpm | 1. | ≥1 min |
| | 2. | 30 bpm ≤ HR ≤ 40 bpm | 2. | ≥5 min |
| Hypotension | 1. | SBP < 91 mmHg | 1. | Two consecutive |
| | 2. | SBP < 70 mmHg | | measurements |
| | | | 2. | At least one |
| | | | | measurement |
| Circulatory | 1. | SBP < 100 mmHg and HR > 110 bpm | 1. | ≥30 min |
| collapse | 2. | SBP < 100 mmHg and HR > 130 bpm | 2. | ≥5 min |
| | 3. | SBP < 100 mmHg and HR < 50 bpm | 3. | ≥30 min |
| | 4. | (Optional) SBP < 100 mmHg and (SpO₂ < | 4. | ≥10 min |
| | | 92% or >5% reduction in SpO₂ from the | | or ≥60 min |
| | | rolling average as defined in relation to | | |
| | | the desaturation subroutine) | | |
| Asystole | | No QRS complexes in the ECG data and | | ≥20 s |
| | | no pulse | | |
| Hypertension | 1. | SBP ≥ 180 mmHg | 1. | ≥60 min |
| | 2. | SBP ≥ 220 mmHg | 2. | At least one |
| | | | | measurement |
| Atrial fibrillation | | Irregular R-R intervals (i.e. different length | | >30 min |
| | | of R-R intervals): Atrial Fibrillation | | |
| | | Regular R-R intervals: Atrial Flutter | | |
| Ventricular | 1. | ≥7 occurrences per minute of | 1. | ≥1 min |
| Extrasystoles | | extrasystoles (n: ≥7), with or without | 2. | When |
| | | normal sinus rhythm interpolated or | | occurring |
| | 2. | ≥3 consecutive occurrences of | | |
| | | extrasystoles, without interpolated sinus | | |
| | | rhythm | | |
| VT/VF | | Unique VT/VF features as detected on | | ≥30 s |
| | | ECG morphology | | |

### System

The present disclosure further relates to a system for automatic detection of a clinical deterioration event in a patient, said system comprising:
one or more sensors configured for automatically monitoring vital sign data (such as heart rate, respiration rate, heart rate variability, temperature, oxygen saturation and blood pressure) of the patient, the one or more sensors further configured for transmitting the vital sign data wirelessly to a server and/or to a gateway;
a first server for receiving and storing the vital sign data, the first server having a computer program thereon, said computer program configured for executing the presently disclosed method thereby providing automatic detection of a clinical deterioration event in a patient; and
one or more gateways configured to provide a wireless communication link between the sensor(s) and the first server.

### Sensors

The sensors to be worn by the patient are preferably selected from the group of: electrocardiography (ECG) sensors, pulse oximeters, oscillometric blood pressure monitors, peripheral skin conductance sensors, 3D accelerometers, peripheral thermometers, and continuous glucose monitors. The sensors are preferably wireless wearable sensors configured for wireless communication with one or more gateways or servers. Data from the sensors may be streamed at a predefined streaming interval in order to save battery consumption and data storage. The streaming interval may be different from sensor to sensor. As an example, the streaming interval for the ECG sensor may be every two minutes, every minute, or every 30 seconds. Furthermore, different time intervals data from each sensor may be selected to be streamed. For example, ECG data may be collected continuously, whereas only 10 seconds of the ECG data may be selected to be streamed each minute. Preferably, the heart rate and temperature of the patient is received continuously. The respiratory rate is preferably received as a 10 second average, which may be streamed continuously or at a predefined interval such as every 10 seconds. The peripheral oxygen saturation and perfusion index is preferably measured (and streamed) every second, and the blood pressure is preferably measured (and streamed) every 15 or every 30 minutes.

### Patient Gateway

A patient gateway should be understood herein as an electronic device configured for communication with one or more sensors and/or servers. An example of a patient gateway is a tablet computer. The gateway is preferably located near the patient, e.g. at the bedside of the patient, such that the wireless signals from the sensors can reach the gateway. The system preferably comprises a patient gateway for each patient. The wireless communication between the sensors and the patient gateway(s) may be any suitable wireless standard such as Bluetooth, Bluetooth Low Energy (BLE), Ultra Wideband (UWB), Wi-Fi, IEEE 802.11ah (Wi-Fi HaLow), GSM, 4G, 5G, or other similar technologies.

### Servers

A server should be understood as a computer or computer program that provides services (e.g. computation) for other programs or devices. The servers of the presently disclosed system is preferably cloud servers, i.e. located remotely from the rest of the system, and accessible through the internet. The presently disclosed subroutines preferably form part of a computer program stored on one or more servers, such as cloud servers. In a preferred embodiment, the computer program comprising the one or more subroutines is stored on the first server. The first server is preferably configured for communication with the patient gateway. Preferably, the communication is encrypted and may be wired or wireless. The wireless communication between the patient gateway and the first server may be any suitable wireless standard as mentioned in relation to the sensors and the patient gateway(s).

The system may further comprise a second server. Preferably, the second server is configured to provide an alarm (e.g. in the form of a push notification) to a remote device (such as a computer, a smartphone or a tablet computer) in case the system has detected a clinical deterioration event or medical complication.

### DETAILED DESCRIPTION OF THE DRAWINGS

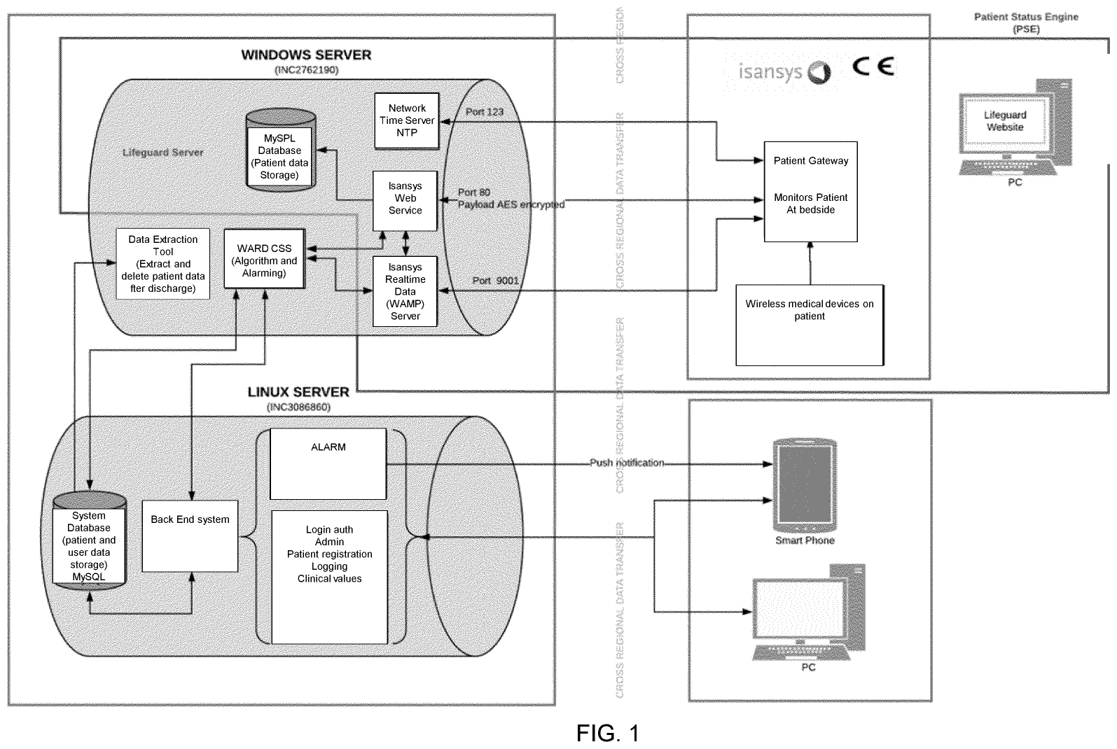

FIG. 1 shows an embodiment of a system according to the present disclosure. In this embodiment, the system comprises one or more wireless sensors, a patient gateway, a first server configured for receiving data from the patient gateway and for executing the disclosed method and subroutines hereof, and a second server for providing an alarm (e.g. a push notification) in case a clinical deterioration event in the patient has been identified. The alarm is preferably transmitted to an external device of the system, such as a smartphone.

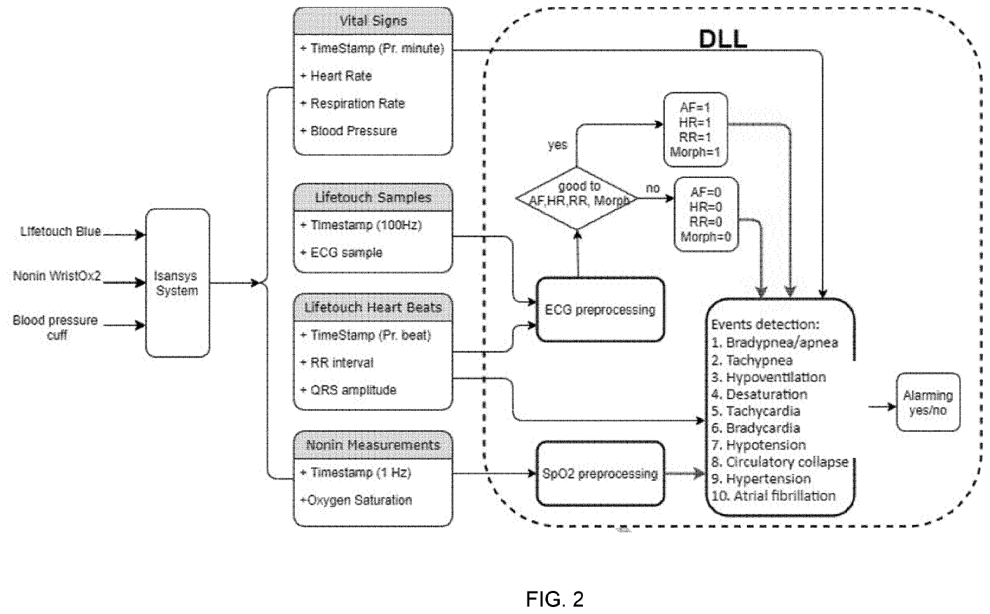

FIG. 2 shows a block diagram, which illustrates the overall functionality of the system and method as disclosed herein. A plurality of vital signs (e.g. heart rate, respiration rate, blood pressure, RR interval, oxygen saturation, etc.) of the patient is received from one or more sensors. Here the system is exemplified with three sensors: A Lifetouch Blue device (combined ECG sensor and accelerometer), a Nonin WristOx sensor (wireless pulse oximeter), and a blood pressure cuff. Some of the vital sign parameters is provided as direct input to the one or more subroutines configured for event detection. Other parameters are provided as input to an ECG preprocessing subroutine configured to assess the quality of the ECG data and/or to a SpO₂ preprocessing subroutine configured to assess the quality of the SpO₂ values received from the pulse oximeter. The ECG preprocessing subroutine assigns a value of either 1 or 0, which indicates the quality of the concerned parameter (e.g. AF, HR, RR, or Morph). A value of 1 indicates that the parameter is good enough to be used in the deterioration event subroutines.

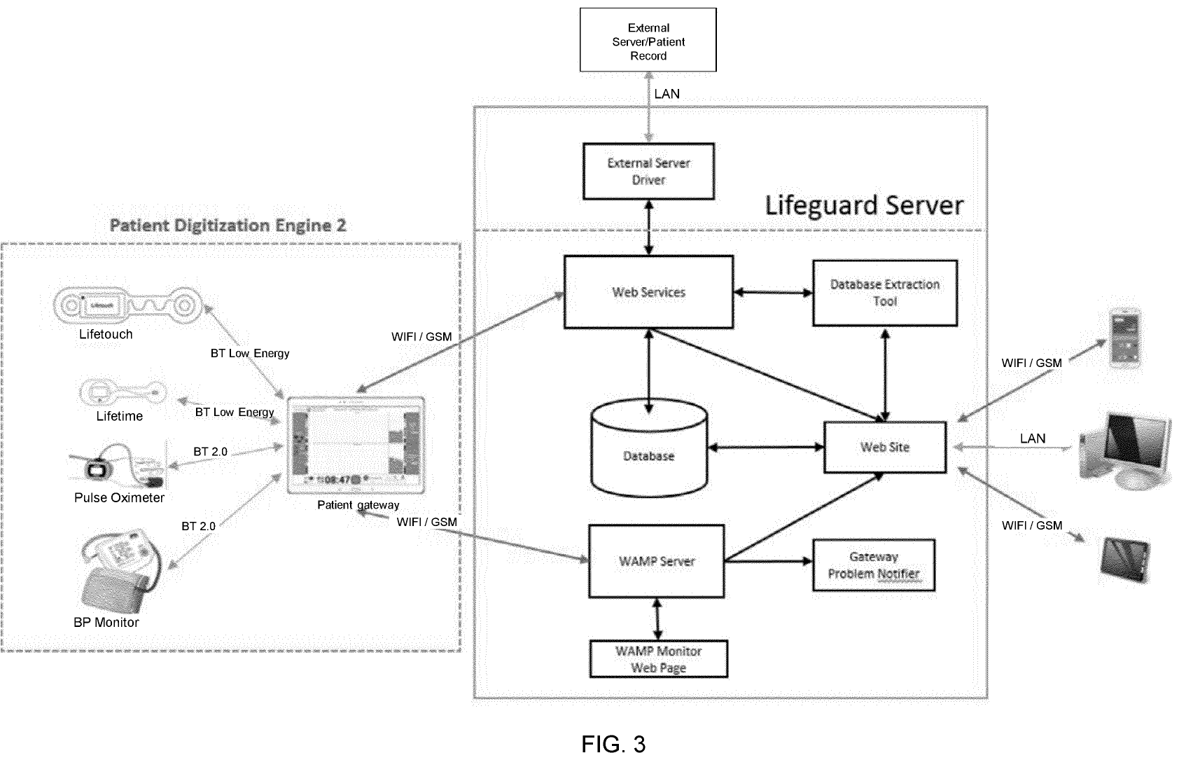

FIG. 3 shows another representation of an embodiment of a system according to the present disclosure. In this embodiment, the system comprises one or more wireless sensors, a patient gateway, a first server (here denoted a Lifeguard server) configured for receiving data from the patient gateway and for executing the disclosed method and subroutines hereof. In this embodiment, the sensors are configured for wireless communication with the patient gateway using Bluetooth or Bluetooth low energy. Furthermore, the patient gateway is configured for wireless communication with the first server using WiFi and/or GSM. The first server may be configured for providing a website, which can be accessed by one or more external devices, such as computers, tablets, smartphones, or similar devices.

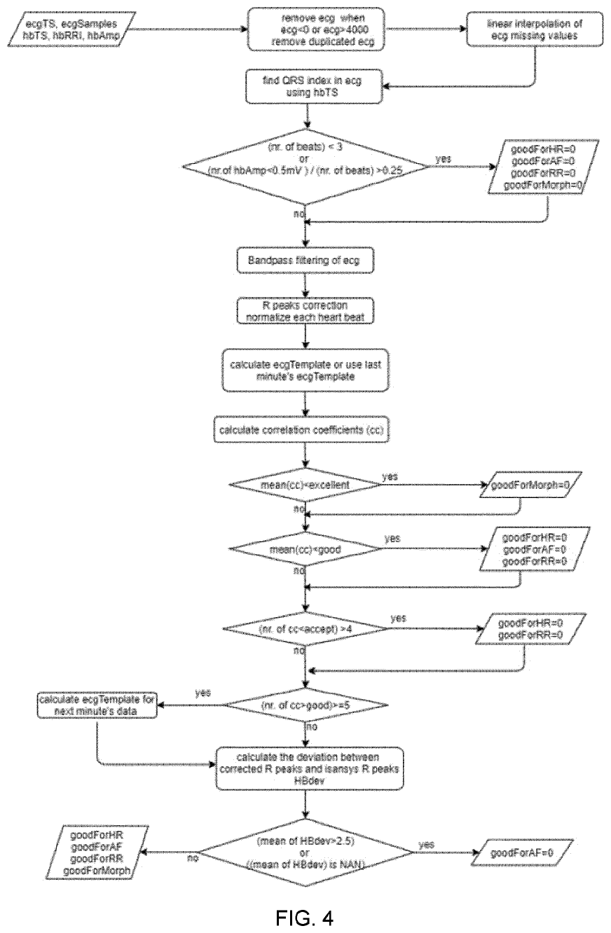

FIG. 4 shows an embodiment of the ECG preprocessing subroutine according to the present disclosure. Generally, a number of vital sign parameters are derived from ECG data received by an ECG sensor. The purpose of the ECG preprocessing subroutine is to assess the quality of the ECG data, such that the derived values (e.g. RRI, HR, and RR) can be validated and used as input in the deterioration event subroutines. The flow diagram shows in detail how the ECG preprocessing subroutine decides whether or not the derived values are good enough to be used as input to the deterioration event subroutines. This is done by performing a series of calculations of the ECG data and by comparing the ECG data and/or calculated values from the ECG data to one or more thresholds, whereby the parameters goodForHR, goodForAF, goodForRR, and godForMorph are assigned a value of 0 or 1.

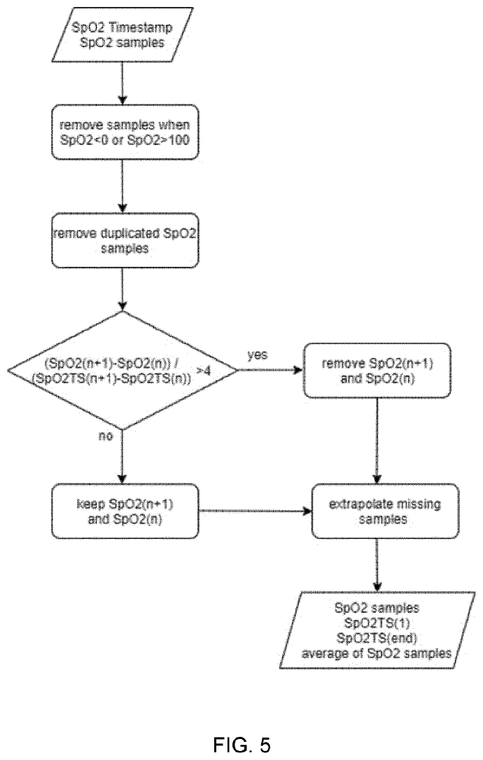

FIG. 5 shows an embodiment of the SpO₂ preprocessing subroutine configured to assess the quality of the SpO₂ values from a pulse oximeter worn by the patient. The purpose of the SpO₂ preprocessing subroutine is to remove SpO₂ values, which are considered unphysical. Examples of unphysical SpO₂ values are SpO₂<0% and SpO₂>100%. Such values are preferably removed from the SpO₂ data. Another example is that the difference of SpO₂ values per second should not exceed 4 percentage points. The SpO₂ preprocessing subroutine is configured to remove such SpO₂ values before calculating an average SpO₂ value.

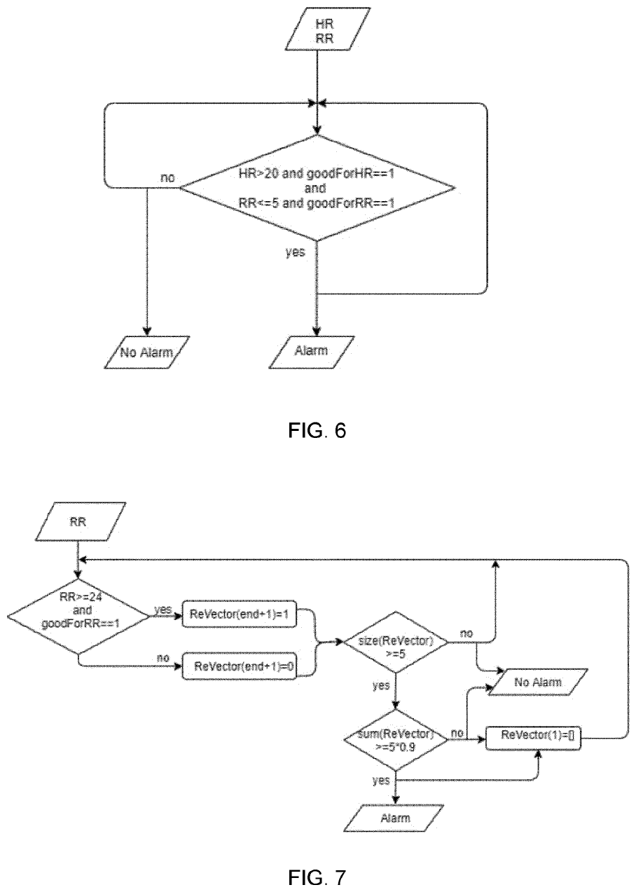

FIG. 6 shows a block diagram of the bradypnea subroutine according to one embodiment. In this embodiment, the bradypnea subroutine is configured for providing an alarm in case HR>20 bpm and RR≤5 bpm for more than 1 minute.

FIG. 7 shows a block diagram of the tachypnea subroutine according to one embodiment. In this embodiment, the tachypnea subroutine is configured for providing an alarm in case RR≥24 for more than 5 minutes.

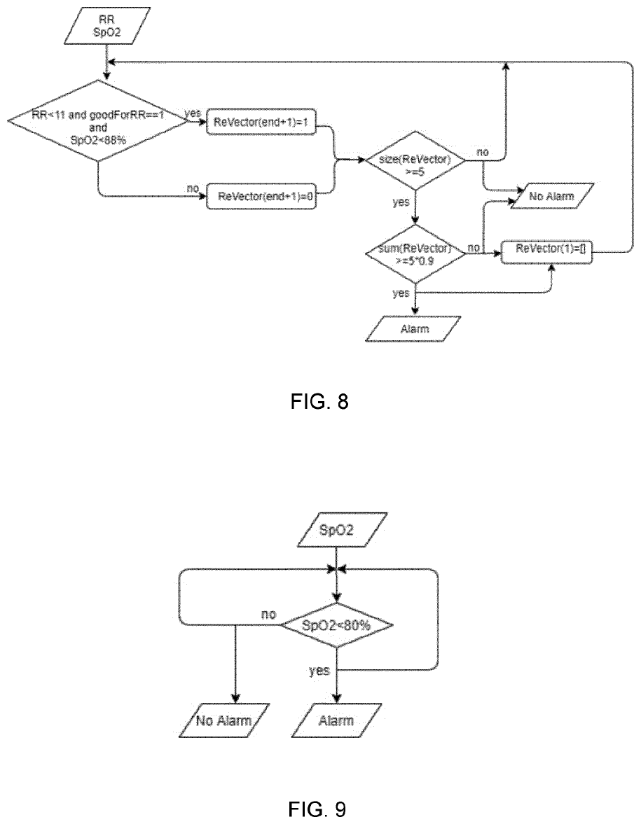

FIG. 8 shows a block diagram of the hypoventilation subroutine according to one embodiment. In this embodiment, the hypoventilation subroutine is configured for providing an alarm in case RR<11 bpm and SpO₂<88% for more than 5 minutes.

FIG. 9 shows a block diagram of the desaturation subroutine according to one embodiment. In this embodiment, the desaturation subroutine is configured for providing an alarm in case SpO₂<80% for more than 1 minute.

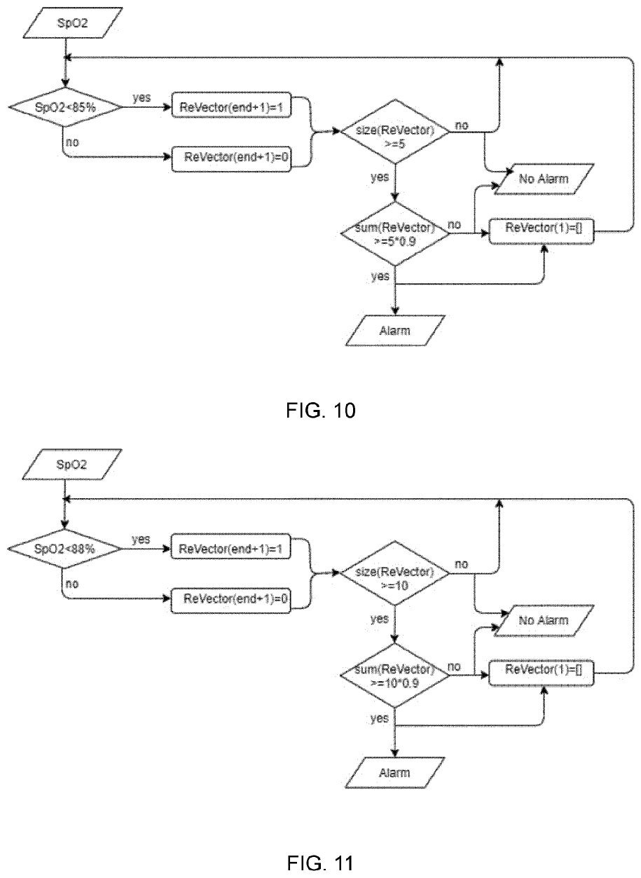

FIG. 10 shows a block diagram of the desaturation subroutine according to one embodiment. In this embodiment, the desaturation subroutine is configured for providing an alarm in case SpO₂<85% for more than 5 minutes.

FIG. 11 shows a block diagram of the desaturation subroutine according to one embodiment. In this embodiment, the desaturation subroutine is configured for providing an alarm in case SpO₂<88% for more than 10 minutes.

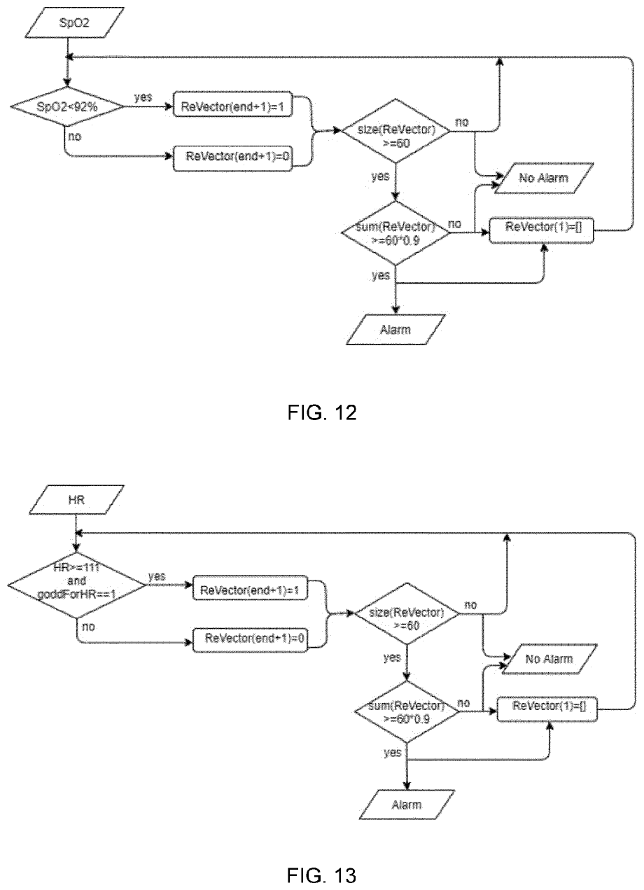

FIG. 12 shows a block diagram of the desaturation subroutine according to one embodiment. In this embodiment, the desaturation subroutine is configured for providing an alarm in case SpO₂<92% for more than 60 minutes.

FIG. 13 shows a block diagram of the sinus tachycardia subroutine according to one embodiment. In this embodiment, the sinus tachycardia subroutine is configured for providing an alarm in case HR≥111 for more than 60 minutes.

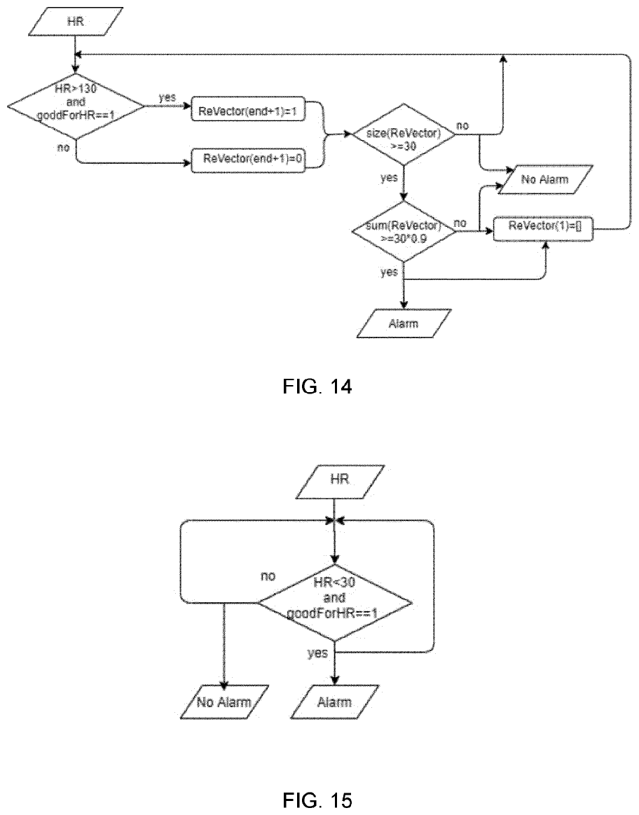

FIG. 14 shows a block diagram of the sinus tachycardia subroutine according to one embodiment. In this embodiment, the sinus tachycardia subroutine is configured for providing an alarm in case HR>130 for more than 30 minutes.

FIG. 15 shows a block diagram of the bradycardia subroutine according to one embodiment. In this embodiment, the bradycardia subroutine is configured for providing an alarm in case HR<30 for more than 1 minute.

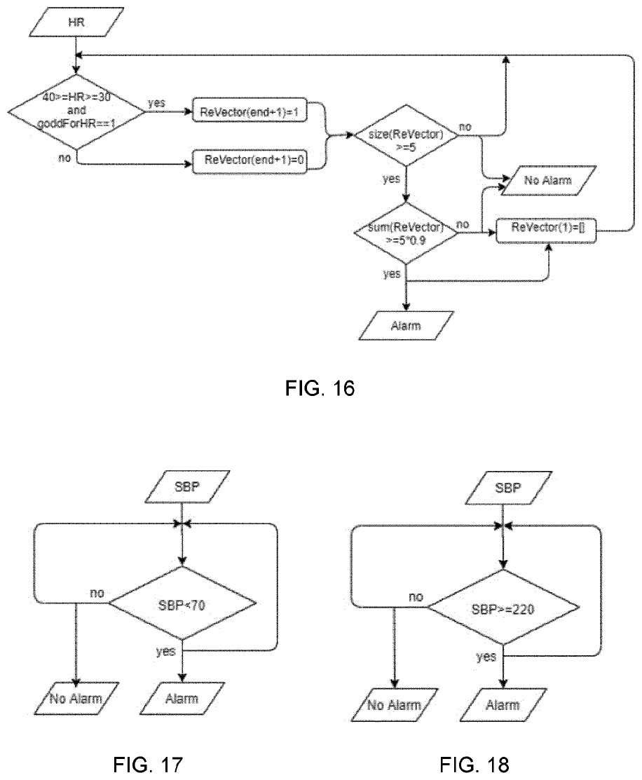

FIG. 16 shows a block diagram of the bradycardia subroutine according to one embodiment. In this embodiment, the bradycardia subroutine is configured for providing an alarm in case 30 bpm≤ HR≤40 bpm for more than 5 minutes.

FIG. 17 shows a block diagram of the hypotension subroutine according to one embodiment. In this embodiment, the hypotension subroutine is configured for providing an alarm in case SBP<70 mmHg.

FIG. 18 shows a block diagram of the hypertension subroutine according to one embodiment. In this embodiment, the hypertension subroutine is configured for providing an alarm in case SBP≥220 mmHg.

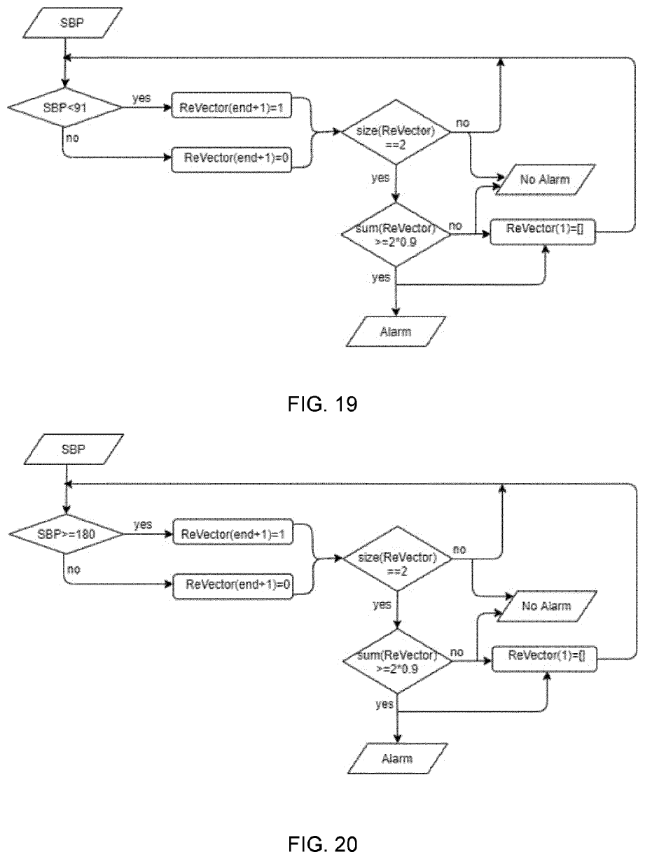

FIG. 19 shows a block diagram of the hypotension subroutine according to one embodiment. In this embodiment, the hypotension subroutine is configured for providing an alarm in case SBP<91 mmHg for two consecutive measurements.

FIG. 20 shows a block diagram of the hypertension subroutine according to one embodiment. In this embodiment, the hypertension subroutine is configured for providing an alarm in case SBP≥180 mmHg for two consecutive measurements.

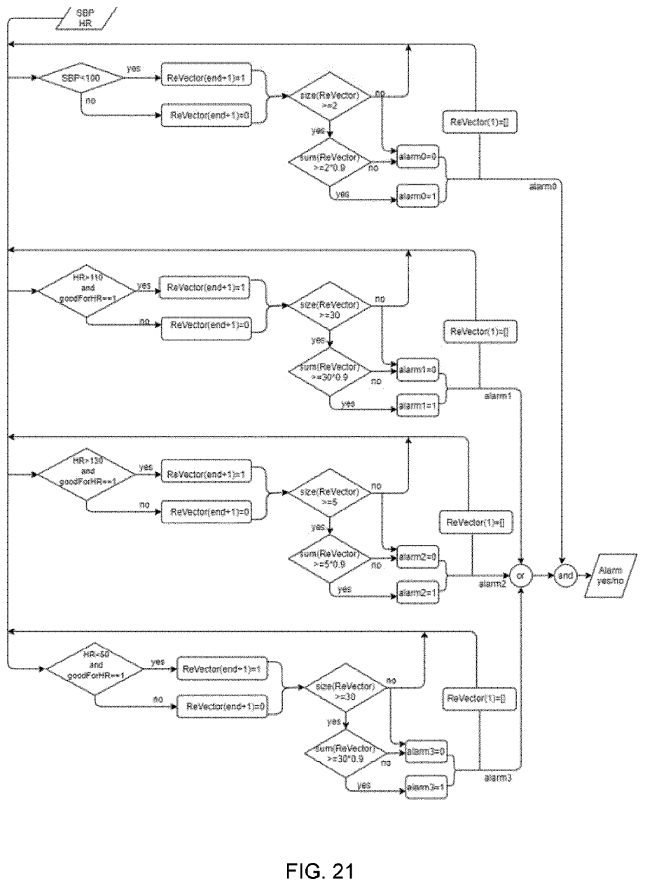

FIG. 21 shows a block diagram of the circulatory collapse subroutine according to one embodiment. In this embodiment, the circulatory collapse subroutine is configured for providing an alarm in case SBP<100 mmHg and HR>110 bpm for t≥30 min, or in case SBP<100 mmHg and HR>130 bpm for t≥5 min, or in case SBP<100 mmHg and HR<50 bpm for t≥30 min.

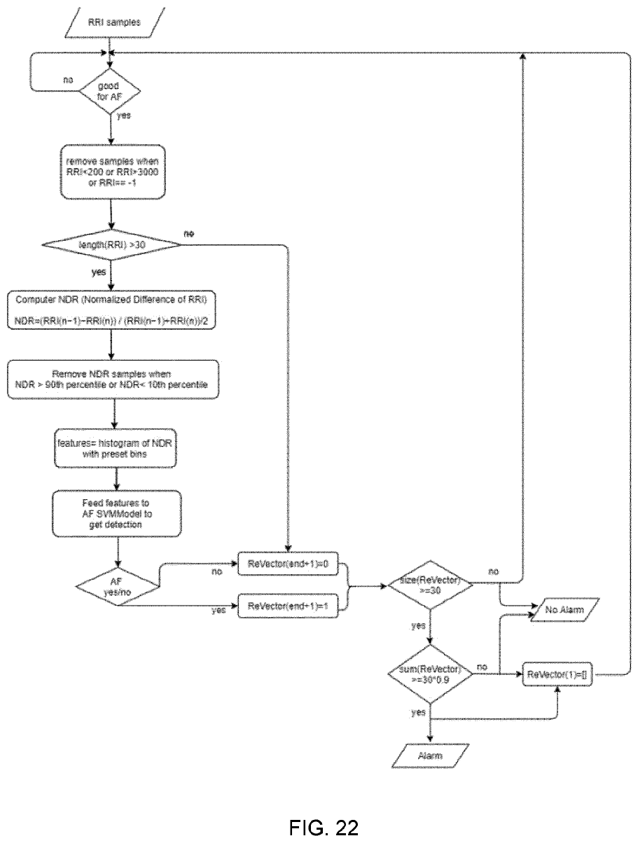

FIG. 22 shows a block diagram of the atrial fibrillation subroutine according to one embodiment. In this embodiment, the atrial fibrillation subroutine is configured for providing an alarm in case of irregular R-R intervals.

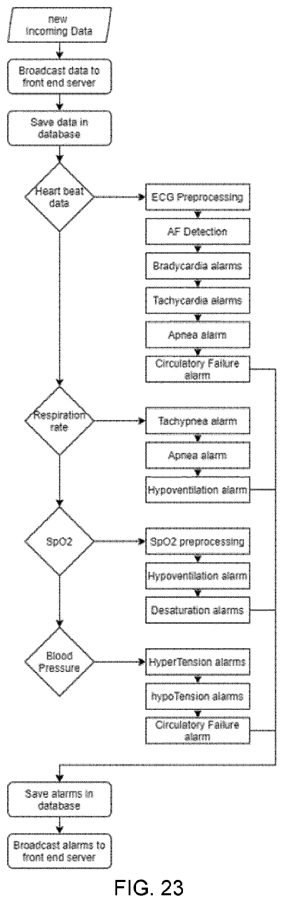

FIG. 23 shows a block diagram, which illustrates the overall functionality of the presently disclosed system and method. The block diagram shows what kind of received data (heart beat data, respiration rate, SpO₂, blood pressure) is used as input to the different subroutines. The deterioration event subroutines are configured to determine whether an alarm should be given based on the received input. The information on alarm (e.g. yes/no) may be saved in a database and subsequently broadcasted to a server or one or more external electronic devices.

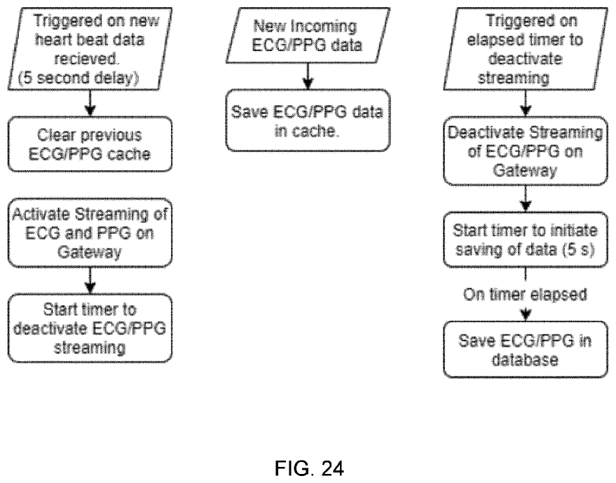

FIG. 24 shows a block diagram, which illustrates how the electrocardiogram (ECG) data and photoplethysmogram (PPG) data is handled. Preferably, the ECG data and PPG data is not streamed continuously as it would consume too much battery on the wireless sensors. Rather, the ECG data and PPG data may be streamed in bundles of data, wherein said stream of data may be started and stopped, e.g. by a timer, during execution of the disclosed method. The streamed ECG/PPG data may be saved in a cache (i.e. memory), which may also be cleared every once in a while, such as when new heart beat data is received.

### Example 1—Detection of Atrial Fibrillation from ECG

Atrial fibrillation (AF) is the most common cardiac arrhythmia and associated with a six times higher risk of stroke, and twice as high risk of death. According to the National Health Service (NHS) AF is the most common heart rhythm disturbance affecting more than 1 million people in the United Kingdom alone. Atrial fibrillation is classified as a tachyarrhythmia, where the electrical impulse is not initiated in the sinus node, but instead in fibrillatory waves in the atrias. Atrial fibrillation may also be characterized as an irregular rhythm with loss of the P-waves in the ECG signal. Preliminary studies have shown that atrial fibrillation is common in post-operative cancer patients. With the presently disclosed approach ECG is available from continuous bedside monitoring thereby providing a possibility of autonomous analysis of the ECG and thereby the possibility to detect atrial fibrillation as demonstrated in this example.

Normally deep neural networks are trained fully supervised, and thus requiring a large amount of labelled data. Vast amounts of medical data exist, but only a small amount of it has been labeled. This can be utilized in semi-supervised learning, where an unsupervised model is jointly trained on large amounts of unlabeled data with a supervised model that is trained on a smaller amount of labelled data. The neural network used in this example is therefore trained in a semi-supervised way where both labelled and unlabelled data is used. This allows for the neural network to learn features from a larger dataset, where the segments are not necessarily labelled. The model is built as a convolutional neural network, utilizing the ResNet architecture.

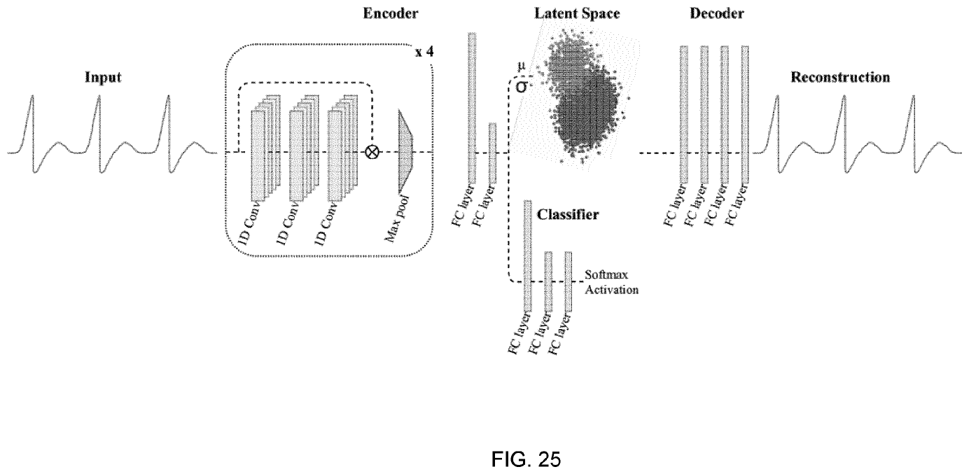

The input to the model is a 10 second segment from single lead ECG. The classification model used after completed training of the model includes the encoder (cf. FIG. 25) and the classifier. The output of the classification model is the probability that the ECG signal from the input segment is showing atrial fibrillation rhythm. The Latent space and the Decoder (cf. FIG. 25), is only used for training the unsupervised part of the model

The data used in this project came from the publicly available MIT-BIH Atrial Fibrillation database (AFDB). The AFDB includes 25 records from different subjects (two only contains the location of the QRS-complexes and no waveform) each of 10 hours length. The remaining 23 records contain the ECG signal obtained from two leads. Each signal was digitized using a sampling frequency of 250 Hz and a 12-bit resolution in the ±10 mV range. Unaudited annotations of the QRS complexes are available along with manual annotation of the into the following subcategories: Atrial Fibrillation, Atrial Flutter, AV-Junctional rhythm and Sinus Rhythm (SR).

Each ECG record was split into 10 seconds non-overlapping segments to avoid the parts of the same segment being present in both the labelled and unlabelled dataset. The label was given based on the annotation files available with the data and was divided into AF vs. Non-AF. In conditions where multiple labels were present in the same segment, the label present for the majority of the segment was used for the entire segment. For both the training and test set, the data was stratified by down-sampling of the majority class. The dataset was split into a training set containing 90% of the segments and a test set containing the remaining 10%. To remove the DC-offset and any baseline wandering before normalization, a high-pass filter with cut off frequency of 0:5 Hz and a filter order of 5. All segments where down sampled to 100 Hz.

The variational autoencoder is a unsupervised generative model, that consists of two neural networks, an inference model, the encoder and a generative model, the decoder. The encoder maps the input sample into a lower dimensional latent variable, which the decoder maps into a reconstruction of the input sample. The variational autoencoder builds upon probability theory and Bayes' rule. In the variational autoencoder the inference model is defined as q-(zjx) and the generative model as p(xjz). By including the label variable, y into the model, a semi-supervised generative probabilistic model can be achieved. In this model the inference model, Q, is defined as q-(zjx; y) q-(yjx), with each term defined as:

$${q}_{ϕ} ( z \vert x , y ) = 𝒩 ( z \vert {μ}_{ϕ} ( x , y ) , diag ( {σ}_{ϕ}^{2} ( x , y ) ) ) , \qquad ( 1 )$$

$${q}_{ϕ} ( y \vert x ) = Bernoulli ( y \vert {π}_{ϕ} ( x ) ) , \qquad ( 2 )$$

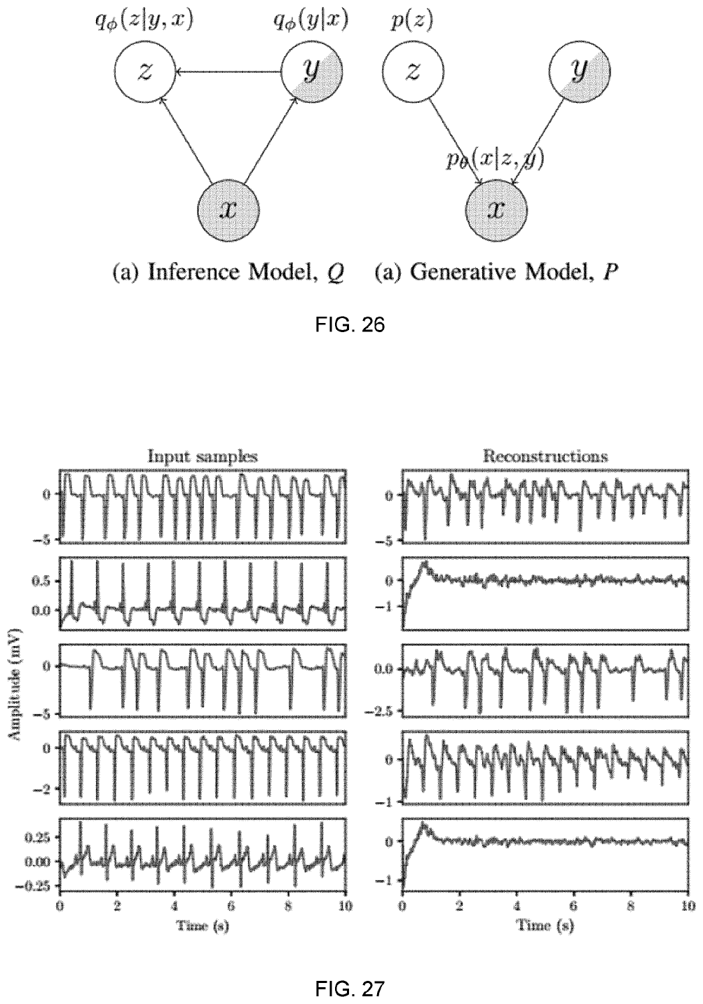

and the generative model, P, is defined as p(z)pΘ(x|z; y), with each term defined as:

$$p ( z ) = 𝒩 ( z \vert 0 , I ) , \qquad ( 3 )$$

$${p}_{θ} ( x \vert z , y ) = f ( x ; z , y , θ ) , \qquad ( 4 )$$

where q_{ϕ} and p_{θ} are neural networks with parameters ϕ and θ, respectively. The inference and generative model is shown in FIG. 26.

The Gaussian distribution q(z|x; y) is achieved by splitting the last layer of the model into two channels representing the mean, μ_{ϕ}, and the log variance, log σ_{ϕ}² of the distributions, from which z is sampled using the reparameterization trick. The reconstruction loss p(x|z; y) is defined as a Gaussian distribution with μ_{θ} being the reconstruction and σ_{θ}²=2.

The objective of optimizing the parameters, θ and ϕ, is to maximize the log-likelihood log p(x). This is achieved by using Jensen' inequality to obtain the evidence lower bound function, which can be optimized. For the unlabeled case the lower bound is given as

$$log p ( x ) = log {\int }_{z} {\sum }_{y} p ( x , y , z ) dz \geq {E}_{{q}_{ϕ} ( z , y \vert x )} [ log \frac{{p}_{θ} ( x , y , z )}{{q}_{ϕ} ( z , y \vert x )} ] ≡ - u ( x ) , \qquad ( 5 )$$

and the labeled case the lower bound is defined as

$$log p ( x , y ) = log {\int }_{z} {\sum }_{y} p ( x , y , z ) dz \geq {E}_{{q}_{ϕ} ( z \vert x , y )} [ log \frac{{p}_{θ} ( x , y , z )}{{q}_{ϕ} ( z \vert x , y )} ] ≡ - ℒ ( x , y ) . \qquad ( 6 )$$

In the lower bounds the contribution of z and y in the unlabeled case and z in the labeled case is marginalized out. For the unlabeled case y is treated as latent variable and is sampled by summing over the two classes, and for z the integral is approximated by sampling from the Gaussian distribution in the latent space. In the case of labeled data, optimization for the labels y is done using binary crossentropy.

Besides the lower bounds defined in equations (5) and (6), an extra loss was introduced where the standard deviations of the input signal and the reconstructions were subtracted and the absolute value was taken of the difference. This was introduced to help the decoder to make better reconstructions. For the classifier, binary cross-entropy loss was used.

To further help the training of the DGM two warmups were introduced, defined as delay and a linear ramp up to a maximum value. One for the KL divergence, with a 25 epoch delay, a max weight of 0.1 at 100 epochs, and a second for the classification loss, with a delay of 0 and a max weight of 0.5 at 40 epochs. These were introduced to not restrain the generative part of the network too much in the beginning, before pushing towards classification and a standard normal distribution for z.

FIG. 25 shows a diagram of the deep generative model used in example 1. 1D Conv: 1-dimensional convolutional layer. FC layer: Fully connected layer.

FIG. 26 shows a diagram of (a) the inference model and (b) the generative model of the proposed network used in example 1. The grey color of the nodes denotes known data, and the partly colored node labelled y emphasized the semi-supervised aspect of the model.

The deep generative model (DGM) can be divided into three parts, the encoder, the classifier and the decoder. The encoder was built with a residual network (ResNet) architecture consisting of four blocks each containing three convolutional layers and a residual connection. ResNet has shown superiority in other image classification tasks, when compared to classic convolutional networks. In order to increase the receptive field of the network, dilation of 2, 4 and 8 was applied to the three layers within each block respectively. Max-pooling was done in the end of each block using a kernel size of 3 and a stride of 3, thus decreasing the signal size by a factor 3 per block. The kernel size and stride were 3 and 1, respectively, for all convolutional layers, and the number of output channels were fixed per block to 32, 32, 64, and 64 for the four blocks respectively. Two fully connected layers was applied to the end of the blocks with a size of 1,000 and 500. The decoder and the classifier were constructed as simple fully connected neural networks (CNN). The decoder consisted of input layer, four hidden layers each with 4,096 nodes and an output layer. The classifier consisted of three layers with 500, 200 and 200 nodes respectively and a binary softmax function as output. All layers except for output layers used Rectified Linear Unit as activation function and had batch normalization and dropout (p=0:3). mA diagram of the model is show in FIG. 2.

In order to demonstrate the potential of using the semi-supervised approach, the proposed DGM was tested against a conventional connected neural network (CNN), identical to the encoder+classifier of the DGM. The setup is constructed using different proportions of unlabeled and labeled data, were the labeled data was used to train both the supervised part of the DGM and the CNN and the unlabeled part of the data was used only to train the unsupervised part of the DGM. In this way a “titration curve” style setup was obtain mimicking cases were different amounts of labeled data could be obtained data. The models was trained in setups using 1%, 5%, 10% and 50% of the data as labeled and the remaining as unlabeled. It was ensured that for each setup, the data in the training and test set was the same for both the DGM and CNN. Furthermore the random seed was fixed such that as much as possible was kept alike between the runs. A total of 111,894 segments were available in the training set after balancing the classes. The test set consisted of 12,434 segments that also were balanced. Each training phase of the DGM consisted of 50 epoch, where labeled data was cycled to correspond with the amount of unlabeled data. As the amount of data per epoch is smaller when training the CNN and thus would lead to fewer updates of the weights if it was only permitted to train for 50 epochs, these were allowed to train for more epoch and instead until convergence.

### Results

The results of the training of the DGM and the CNN using different amounts of labeled data is shown in the table below.

| No. of labelled | DGM | | | CNN | | |
|---|---|---|---|---|---|---|
| data | Acc | Sen | Spe | Acc | Sen | Spe |
| 1% (1,118) | 94.0% | 98.8% | 89.3% | 65.8% | 93.6% | 38.0% |
| 5% (5,594) | 98.7% | 98.5% | 98.9% | 95.3% | 98.5% | 92.1% |
| 10% (11,190) | 98.7% | 98.9% | 98.5% | 97.7% | 96.2% | 99.1% |
| 50% (55,948) | 98.8% | 98.9% | 98.8% | 98.2% | 97.5% | 99.0% |

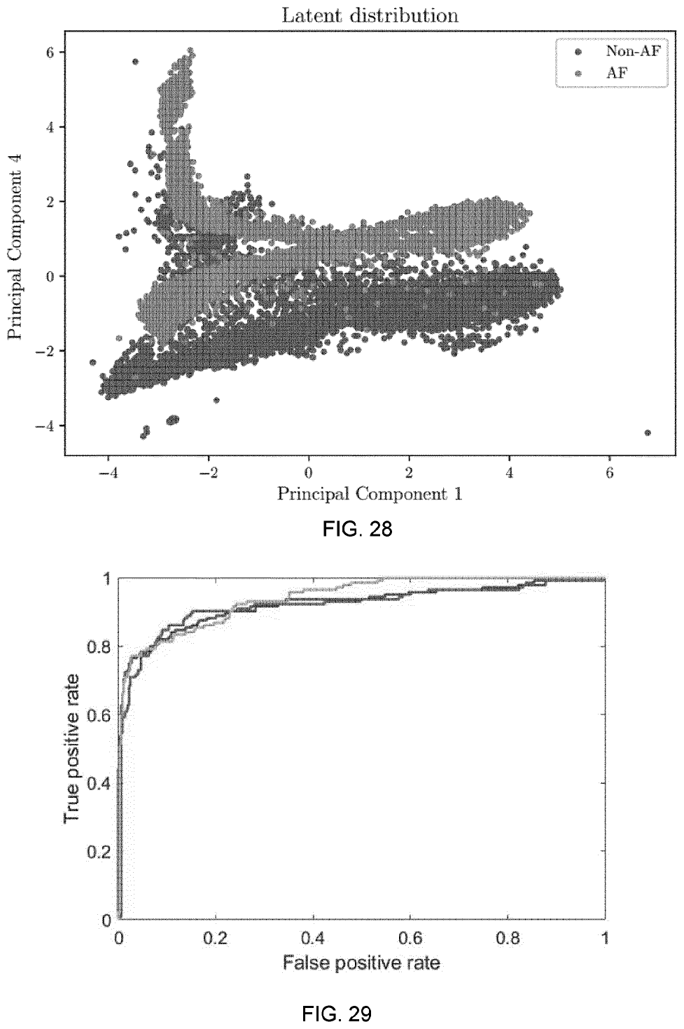

The best result is obtained by the DGM in the semi-supervised approach using 50% of the data labeled. The input segment and corresponding reconstruction of chosen samples is shown in FIG. 28, and the distribution of the samples for the test set in the latent space is shown in FIG. 29. The results show a maximal performance of the model on the stratified test set of 98.8% with a sensitivity of 98.9% and a specificity of 98.8%. This was obtained using 50% of the training data as labelled data and 50% as unlabeled data. The results in the table above show that the proposed semi-supervised approach achieves higher performance in all test cases, with the most prominent difference in the cases with lower amount of labeled data. Comparing the results at different amounts of labeled data even shows, that the semi-supervised approach with 5% labeled data is superior to the highest obtained performance by the fully-supervised approach in the 50% labeled data. Even in the case of 1% labeled data, the DGM achieved an accuracy of 94.0%.

### Example 2—Prediction of Serious Adverse Event (SAE) from Vital Signs

In general monitoring post-operative patients is important for preventing serious adverse events (SAE), which increases morbidity and mortality, but currently monitoring of post-operative patients relies on intermittent bedside monitoring. The presently disclosed approach facilitates continuous and predictive monitoring and therefore improves the management of patients. This example demonstrates machine learning based prediction of SAE in post-operative patient based on vital signs acquired by wearable sensors showing that SAEs can be predicated with high AUROC of as high as 93% by monitoring only four common vital signs. Using descriptive statistics extracted from trends as features and SVM based machine learning technique, as in this example, reduces algorithm complexity and thereby consume less battery power, which is very important for wearable systems.

This example demonstrates classification of “SAE” versus “no SAE” in 2 hours (prediction window) based on last 10 hours recordings (observation window). First, the trends of time series of vital signs were extracted with moving average in order to remove noises. Then the descriptive statistics were calculated from the trend of each modality and concatenated into a feature vector. Finally, a machine learning based on support vector machine was employed for prediction of SAE.

During the study the vital signs of heart rate, respiration rate, and blood oxygen saturation, were continuously acquired by wearable devices and blood pressure was measured intermittently from 453 post-operative patients. Data acquisition was managed by the Isansys patient status engine.

The study took place at Rigshospitalet and Bispebjerg Hospital in Copenhagen, Denmark from February 2018 to August 2020. 453 post-operative patients (278 males, 175 females) were included in the study. The average age was 71 years (range: 60-93) and the average amount of monitoring hours was 79 hours (range: 0.73-168.8). Patients in the study had a wide range of clinical SAEs ranging from neurologic, respiratory, circulatory, infectious and other complications. Information about SAEs were registered by medical doctors.

The vital signs HR, RR and SpO2 were acquired continuously by the wearable sensors and BP was measured intermittently. The acquisition of vital signs was managed by Isansys patient status engine (PSE) (Isansys Lifecare Ltd). The Isansys Lifetouch was attached to the patients' chest for acquiring single lead ECG with a sampling frequency of 1000 Hz, from which HR in beats per minute and RR in breaths per minute were derived. Pulse Oximeter (Nonin Model 3150 WristOx2) was attached to the finger for the acquisition of the photoplethysmogram (PPG) with sampling frequency of 75 Hz, from which SpO₂ as a percentage was derived. The wearable sensors' data and derived values were first transmitted via Bluetooth to a gateway of PSE, which was located near the bed of the patient, and then to a hospital server for storing data in a patient database via WIFI every minute. Systolic blood pressure (sysBP) in mmHg was measured intermittently by using Meditech BlueBP-05. These sysBP measurements were entered into the gateway by medical staff and then automatically transmitted to the patient database. HR, RR, SpO2 and sysBP were synchronized through their timestamps.

### Prediction of Serious Adverse Events

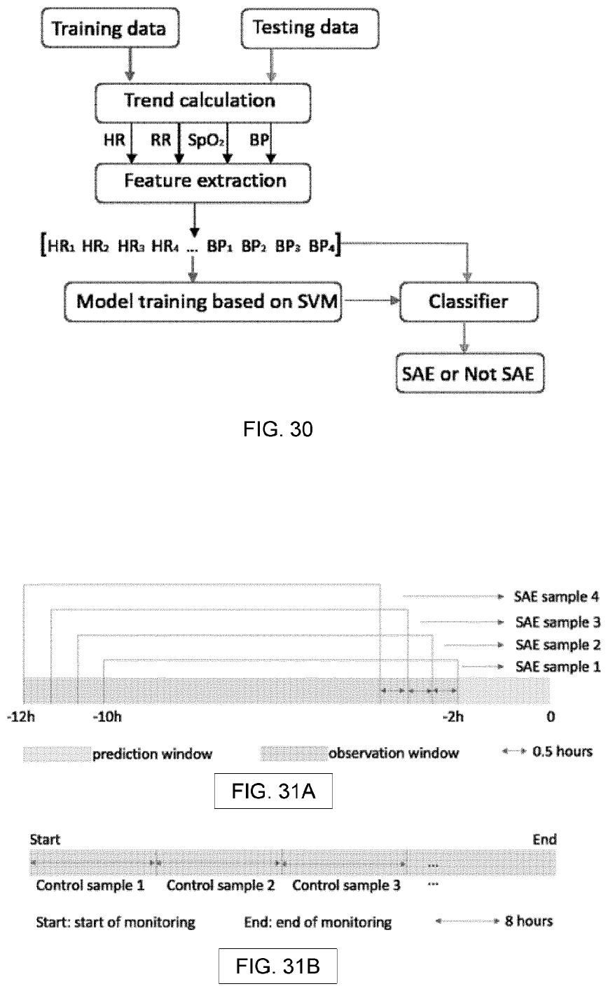

Prediction of SAE can be seen as a classification problem aiming to classify “SAE” versus “no SAE” over a time period (prediction window), e.g. few hours, based on last recordings (observation window). The prediction window was chosen to be two hours and the observation window was chosen to be ten hours as shown in FIG. 31A. In this study samples of SAEs resulting from neurologic, respiratory, circulatory, infectious and other complications were extracted from the patients' database. These extracted SAEs' samples were regarded as “SAE class”. The control class' samples were extracted from patients who didn't have SAEs during the monitoring period. A classifier for prediction of SAE was trained from those two classes. The prediction of SAE was based on the features extracted from trends of four time series HR, RR, SpO₂ and sysBP and on a classification carried out with a support vector machine (SVM) model. FIG. 30 depicts the steps of the algorithm applied.

1) Extraction of SAE class and control class: The SAE class was identified based on SAEs' timestamps. To account for class imbalance, the SAE class was oversampled. SAE class samples were extracted as eight hours' time series of vital signs with overlapping from two hours before to twelve hours before SAE timestamp. Four samples were extracted for each SAE as illustrated in FIG. 31A. Control class samples were extracted from patients who did not have SAEs during vital sign monitoring at the hospital and the monitoring duration was at least eight hours. FIG. 31B illustrates the extraction of control samples. The samples were extracted during the whole monitoring period to cover all possible patients' statues.

2) Feature extraction: Selection of discriminative features is normally important for the prediction of SAE. One or more clinical deterioration events are often preceded with SAE and can be extracted from vital signs as demonstrated in the presently disclosed approach. In this example the trends of time series of HR, RR, SpO2 and sysBP were extracted by using moving average with a sliding window of 60 minutes. In this examples the trends were supposed to represent the deterioration. Then four descriptive statistics (maximum, minimum, mean, and standard deviation) were calculated from the trend of each modality as features. The features from each modality were concatenated into one feature vector. The size of each feature vector was sixteen.

3) SVM classification: The SVM model used in this example is a supervised machine learning algorithm for solving classification and regression problems. It has shown good generalization property in many applications. The basic idea is to construct an optimal hyperplane for linearly separable patterns. The optimal hyperplane is the one that has maximal margin between two classes. For the non-linearly separable patterns one solution is to transform original data into a higher or indefinite dimensional space and then find a separating hyperplane in the transformed space by using a kernel function. Given a training set (xᵢ; yᵢ), i=1, . . . , N where xᵢ∈Rⁿ and yᵢ={±1}, xᵢ is a data point and y; indicates the class which the point x; belongs to. The output of the classifier is defined as

$$y ( {x}_{i} ) = sign [ {w}^{T} φ ( {x}_{i} ) + b ]$$

where the function' maps xᵢ into a higher dimensional space. w is the weight vector and b is the bias of the hyperplane. The standard SVM requires the solution of the following optimization problem:

$${min}_{w , b , ξ} \frac{1}{2} {w}^{T} w + c {\sum }_{i = 1}^{N} {ξ}_{i}$$

subject to

$$\{ \begin{matrix}{y}_{i} ( {w}^{T} φ ( {x}_{i} ) + b ) \geq 1 - {ξ}_{i} , & i = 1 , ... , N \\ {ξ}_{i} \geq 0 , & i = 1 , ... , N\end{matrix}$$

where ζᵢ is a slack variable and c is a penalty parameter. They are introduced if the training data cannot be separated without error. As a consequence, training samples can be at a small distance ζᵢ on the wrong side of the hyperplane. In practice, there is a trade-off between a low training error and a large margin. This trade-off is controlled by the penalty parameter c. A Gaussian kernel k was chosen for non-linear SVM classifier in this study:

$$k ( {x}_{i} , {x}_{j} ) = exp ( - \frac{{‖ {x}_{i} - {x}_{j} ‖}^{2}}{2 {σ}^{2}} ) = {φ ( {x}_{i} )}^{T} φ ( {x}_{j} )$$

where σ is the width of Gaussian kernel. Tuning of σ is important for optimizing classifier performance. Threefold cross-validation was applied to estimate the classification performance. The misclassification cost (N_{SAE}+n_{control})/n_{SAE} was given to SAE data samples, whereas (n_{SAE}+n_{control})/n_{control} to control data samples. Here, n_{SAE} and n_{control} represent the number of data samples belonging to SAE class and control class, respectively. The dataset was randomly partitioned into three subsets. One subset (a testing set) was used to validate the classifier trained on the remaining two subsets (a training set). This process was repeated three times such that each subset was validated once. During training, the training set was further divided into subsets for optimizing Gaussian kernel parameter σ and boxconstraints (inner cross-validation). The set of parameters, boxconstraints and σ, were searched among positive values, with a log-scale in the range [10⁻³; 10³]. The optimal boxconstraints and σ were then applied to build classifier for the testing set. The performance of the classifier was evaluated in terms of sensitivity, specificity, positive predictive value (PPV), negative predictive value (NPV) and the area under receiver operating characteristic curve (AUROC).

TABLE I
The performance of classifier from threefold cross validation

| | ACC. | SEN. | SPE. | PPV | NPV | AUROC |
|---|---|---|---|---|---|---|
| Test 1 | 88.91% | 80.00% | 92.41% | 80.56% | 92.16% | 92.47% |
| Test 2 | 89.28% | 82.75% | 91.85% | 80.00% | 93.11% | 91.96% |
| Test 3 | 90.25% | 78.62% | 94.84% | 85.71% | 91.84% | 94.12% |
| Average | 89.48% | 80.46% | 93.03% | 82.08% | 92.37% | 92.86% |

(ACC = Accuracy; SEN = Sensitivity; SPE = Specificity)

### Results

The performance of the classifier with threefold cross validation was summarized in Table I. The accuracy, sensitivity, specificity, PPV, NPV and area under receiver operating characteristic curve (AUROC) are relatively close among three tests. The classifier achieved an averaged accuracy of 89%, sensitivity of 80%, specificity of 93%, PPV of 82%, NPV of 92% and AUROC of 93%. Additionally, FIG. 29 shows the receiver operating characteristic curves (ROC) for three tests. The averaged AUROC of 93% indicates good discriminative power of the classifier. These findings are promising and demonstrate the feasibility of predicting SAE from vital signs acquired with wearable devices and intermittent measurement.

### Example 3—Non-Invasive Blood Pressure Estimation Based on Vital Signs

This example demonstrates a new non-invasive way of estimating blood pressure (BP) of patients without the need for the normal cuff. It is based on measured vital signs, as also disclosed herein, and application of artificial intelligence, in particular a trained machine learning model. The new BP estimation doesn't require the usual strict synchronization between wearable devices. As vital signs can be acquired continuously and in real-time, the presently disclosed BP estimation can also be provided in real time, for example by the application of a trained machine learning model.

Blood pressure (BP) is a key hemodynamic variable for the evaluation and diagnosis of conditions such as stroke and cardiovascular disease. BP can vary dramatically from beat to beat, and minute to minute. It is crucial to monitor BP continuously on post-operative patients. Currently BP is often monitored continuously with an invasive arterial catheter, for example in critically ill patients in the ICU. This way has risk of infection and need clinical operation. Outside ICU, BP is measured by a cuff-based device, however only intermittently. The inflation/deflation often causes discomfort/pain for the patients and disturbs their rest. The cuffless BP estimation is therefore favored. Many cuffless BP estimations are based on features which require synchronization between electrocardiogram (ECG) and photoplethysmogram (PPG). Since ECG and PPG are recorded from two different devices, synchronization between ECG and PPG often causes problem. In his study, we propose a new way to estimate BP based on vital signs heart rate (HR), respiration rate (RR), blood oxygen saturation (SpO₂) and pulse rate (PR). The vital signs are calculated independently and not sensitive to the synchronization.

### Methods

498 post-operative patients participated in the study. After major abdominal cancer surgery, they were re-admitted to the general ward where their vital signs were monitored for up to four days, with the approach described herein. Severe adverse events, resulting from a wide range of complications, were collected for up to 30 days. Two wearable devices were attached to the patients for acquiring vital signs continuously. One was the Isansys Lifetouch at chest for acquiring single lead ECG, from which HR and RR were derived. Another was a Pulse Oximeter at the finger for acquiring PPG, from which SpO₂ and PR was derived. Systolic blood pressure (SBP) and diastolic blood pressure (DBP) was measured intermittently by Meditech BlueBP-05. The wireless acquisition and transmission of vital signs was managed by Isansys patient status engine (PSE) (Isansys Lifecare Ltd). HR, RR, SpO2, PR, SBP and DBP were synchronized through their timestamps.

Random forest with 200 trees was applied for estimation of DBP and SBP, but other models can be used. First, 3 hours' time series of HR, RR, SpO2 and PR before BP measurements were extracted, from which descriptive statistics such as mean, standard deviation and range were calculated as features. Then, the regression model was trained with first day's data of each patient. The trained model was tested by the following days' data. The mean absolute error (MAE) and standard deviation (STD) of the error were used for evaluating estimation performance.

### Results

Estimation performance is shown in the Table I. According to Association for the Advancement of Medical Instrumentation (AAMI) standard, MAE should be less than or equal to 5 mmHg and STD should be less than or equal to 8 mmHg for both DBP and SDP. In this example STD of DBP met the standard and MAE was close to the standard. STD of SBP is closer to the standard, while MAE is higher

TABLE
Blood pressure estimation

| 498 patients | MAE (mmHg) | STD (mmHg) |
|---|---|---|
| DBP | 6.01 | 4.98 |
| SBP | 10.53 | 8.44 |

### Example 4—Prediction of SAE from Night-Time Vital Signs

The period directly following surgery is critical for patients as they are volatile to infections and other types of complications, i.e. severe adverse events (SAE). Impending complications might alter the circadian rhythm and, therefore, be detectable during the night before. This example provides a prediction model that can classify nighttime vital signs depending on whether they precede a serious adverse event or come from a patient that does not have a complication at all, based on data from 450 post-operative patients. The prediction model is compared to random classifiers to demonstrate the applicability.

Circadian clocks, which are autonomous molecular mechanisms, are found in all mammalian cells and regulate body functions, such as hormone secretion, immune response and the sleep/wake phases. These normal changes in cardiovascular function can be accompanied by adverse events, as, for example, the onset of myocardial infarctions or sudden cardiac death has been found to be elevated in the early morning compared to nighttime. Antiarrhythmic mechanisms, such as increasing heart rate variability, can be constrained by disease and, therefore, protection might not be optimal. As demonstrated herein serious adverse events might be preceded by changes in vital signs during the night, stemming from deactivation of the patients' parasympathetic nervous system. Because heart rate and respiration rate reliably reach their nadir during sleep, nighttime offers an opportunity to observe the physiological baseline and make a comparison between patients that will have a complication and patients that will not. This might cause an increase in heart rate, respiratory rate and blood pressure.

### Vital Signs Monitoring

Vital signs were obtained according to the procedure as described herein. In particular systolic (SBP) and diastolic (DBP) blood pressure values in mmHg were automatically recorded using the Meditech BlueBP-05. Heart rate (HR) and respiratory rate (RR) were obtained once per minute from the Isansys Lifetouch single lead ECG using a sampling rate of 1000 Hz at the chest of the patient. The pulse rate measurements were taken at the patient's arm. Photoplethysmogram (PPG) was measured at a rate of 75 Hz using a pulse oximeter (Nonin Model 3150 WristOx2). The SpO₂ values were determined from that.

### Modelling

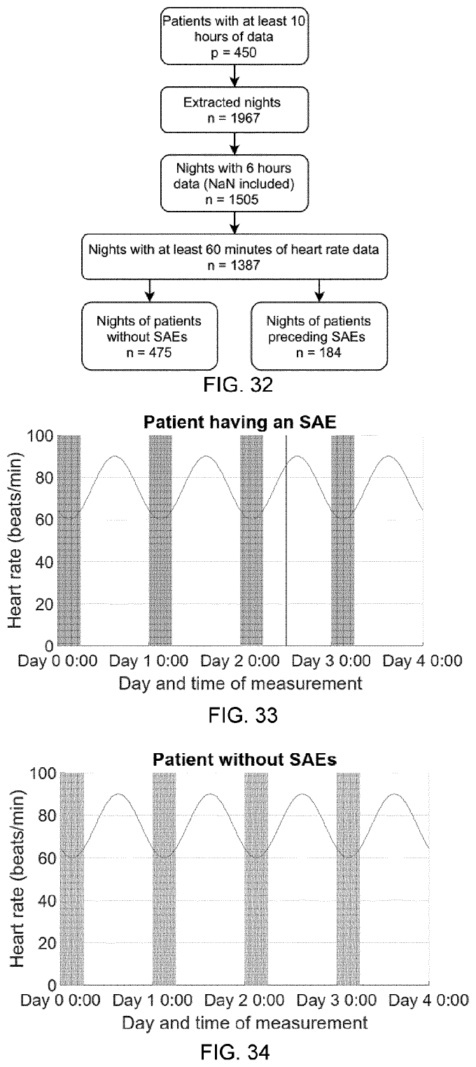

The problem was modelled as a binary classification task. The nights, which were defined as ranging from midnight to 6 AM, were extracted from the continuous measurements and either labelled as 1, if they preceded a SAE or 0, if they did not. FIG. 33 illustrates the extraction process, on the basis of an idealized four day heart rate measurement. As shown, heart rate usually decreases during the night and rises during the day. SAEs were always allocated to the day they were recorded and nights, during which an SAE happened, were discarded. If data collection started or ended during a night, it was also rejected. To ensure that a minimum of usable data was present, at least 60 valid data points for heart rate values were required in a night. This modality was chosen after consultation with clinicians as they were expected to have a degree of predictive validity. The process is illustrated in FIG. 32.

### Feature Extraction

The procedures described in the previous sections provide one 360 minutes×6 modalities vector for each night. For each modality with at least one recorded datapoint, missing values are filled by performing first forward and then backward-carry. In order to smooth the data and correct for measurement errors, the moving average is computed using the nearest 10 values. For each night, 9 features are calculated: mean, median, standard deviation (STD), maximum, minimum, kurtosis, skewness, 10^{th} and 90^{th} percentile. The mean, median and standard deviation can be useful to detect anomalous vital sign values such as an elevated heart rate or an unstable respiration rate. The maximum and minimum measured during the night can indicate unusual events such as hypoxemia episodes in case of SpO2. Kurtosis measures the weight of a distribution's tail relative to the center, skewness evaluates its asymmetry. The 10^{th} and 90^{th} percentile provide information about the distribution. At each time steps, the static variables age, gender, height, weight, whether the patient smokes, number of packs smoked and units of alcohol consumed per week are added. This results in a total of (9*6)+7=61 features. Missing features, which arise if not a single value was recorded for the respective modality during the whole night, are filled by mean imputation.

### Classification

After feature extraction, 5-fold cross validation is used to split the data into a training set and a test set. Each fold is used once for testing while the four remaining folds constitute the training set. This procedure is performed 10 times and from the evaluation metrics the average is computed. To correct for the imbalance in the dataset, the Synthetic minority Oversampling Technique (SMOTE) is applied to the training but not the test set. The implementation used in this example was provided by the imbalanced-learn library and brings both classes to equal size. XGBoost was chosen as the classification algorithm, because XGBoost has shown to deliver state-of-the-art performance while running faster than most other solutions. In short, the algorithm works via gradient tree boosting. Given a dataset D={(xᵢ,yᵢ)}(|D|=n,xᵢ∈ [symbol] ^{m},yᵢ∈( [symbol] ), with n examples and m features, the output is predicted by adding over K functions

$${\hat{y}}_{i} = ϕ ( {x}_{i} ) = {\sum }_{k = 1}^{K} {f}_{k} ( {x}_{i} ) , {f}_{k} ∈ F .$$

In the formulation above

$$F = \{ f ( x ) = {w}_{q ( x )} \} ( q : {ℝ}^{m} -> T , w ∈ {ℝ}^{T} )$$

and represents the space of trees, q is the structure of the trees and T the number of leaves per tree. From this representation, the regularized objective function can be derived:

$$L ( ϕ ) = {\sum }_{i} l ( {\hat{y}}_{i} , {y}_{i} ) + {\sum }_{k} Ω ( {f}_{k} )$$

$$with$$

$$Ω ( f ) = γ T + \frac{1}{2} λ {‖ w ‖}^{2}$$

where ŷᵢ is the prediction, yᵢ the true outcome and I the loss function. To prevent the model from overfitting, a regularization function xan be included as a second term. Because all the trees cannot be learned at once, the model parameters are learned in an additive fashion. In the formula only the functions ft that optimize the model are chosen.

This prediction approach can be compared to two implementations of a random classifier as provided by scikit-learn's Dummy classifier class. The first, uniform version simple represents a coin flip and chooses the classes with equal probability. The second, stratified version chooses the classes with the same probability as presented to the classifier in the labelled training output sets, so the majority class if chosen more frequently.

### Results

To assess the effectiveness of this approach, standard performance measures were calculated. The accuracy shows which percentage of the test data was predicted correctly, recall, precision and F1-score provide information about the type of errors. Additionally, the ROC-AUC value gets computed by integrating over the area below the curve.

After the data was filtered as described in the previous section, 184 nights preceding SAEs and 475 nights of patients without SAEs remained. In the table below, the mean values per night for both classes are shown, with the standard deviation of the means in brackets. On average, on nights preceding SAEs, patients have a higher heart and breathing rate as well as a slightly lower oxygen saturation compared to the patients without complications. However, it is also evident that due to the large standard deviation a simple threshold-based classifier is not sufficient to solve this task, which reaffirms the presently disclosed machine learning based approach.

TABLE
Mean night modality values for both groups

| | SpO2 | HR | RR | PR | DBP | SBP |
|---|---|---|---|---|---|---|
| SAE nights | 92.06 | 79.00 | 15.41 | 79.60 | 64.38 | 121.28 |
| | (3.42) | (15.87) | (3.15) | (16.93) | (11.57) | (23.15) |
| Non-SAE | 92.66 | 72.31 | 13.96 | 72.88 | 64.92 | 124.80 |
| nights | (3.03) | (12.72) | (2.58) | (16.72) | (11.46) | (21.21) |

Comparing the average percentage of missing data in the table below, it can be seen that the SAE group has many more values missing than the Non-SAE group. A reason for that could be that very sick patients take off their measurement devices more frequently, especially the oxygen saturation sensor at the finger.

TABLE
Mean percentage of missing values for both groups

| | SpO2 | HR | RR | PR | DBP | SBP |
|---|---|---|---|---|---|---|
| SAE nights | 42.22 | 2.29 | 3.05 | 43.12 | 51.00 | 51.00 |
| Non-SAE nights | 30.2 | 1.96 | 1.9 | 33.45 | 38.93 | 38.93 |

### Performance of the Complication Prediction Model

The table below present the performance of the classifier compared to the two random baseline models. The model of this example achieved a F1-score of 0.49, a precision of 0.58, an accuracy of 0.75 and a ROC-AUC score of 0.65, all better than baseline. However, this classifier underperforms on the recall metric, which is due to the wide standard deviations as presented earlier.

TABLE
Classifier performance from 5-fold cross-validation

| | F1 | Recall | Precision | Accuracy | ROC-AUC |
|---|---|---|---|---|---|
| XGBoost | 0.49 | 0.43 | 0.58 | 0.75 | 0.65 |
| Uniform | 0.36 | 0.53 | 0.27 | 0.47 | 0.49 |
| Stratified | 0.37 | 0.49 | 0.29 | 0.53 | 0.52 |

### Summary

The present example has some limitations: All the data used came from a single cohort for both training and validation. A model trained on data from various institutions will generalize better and be more valuable in clinical contexts. Additionally, the fact that the SAEs used as outcome measures in this study have different causes and severities and might lead to changes in the vital signs at all, could explain the heterogeneous results. There were also notable differences between the nights which preceded an SAE and the nights which did not in terms of missing data. Nights before a critical event had a higher percentage of data missing for all modalities. Another issue is that it was assumed that patients were sleeping based on the time of day. If a patient is awake during the night their vital signs could be altered and make prediction more difficult. Combining the algorithm in this example with a sleep stage detector, for example based on EEG measurements, could substantially improve its predictive capabilities. In spite of these limitations this example further illustrates that monitoring of vital signs is an important tool for prediction of SAEs, and that nighttime monitoring can further improve the prediction of SAEs, possibly even hours before the SAE arises, and the presently disclosed approach provided a significant step in the understanding of disease progression during sleep.

### Patient Data

The data for this project was acquired at Rigshospitalet and Bispebjerg Hospital in Copenhagen, Denmark from February 2018 to August 2020. The 450 patients (275 males, 175 females) had a mean age of 71 years (range 60-93). On average there were 80 hours of data recorded (range 12-169). A serious adverse event (SAE) as defined according to the guidelines as any medical occurrence that results in death, results in in subject hospitalization, results in persistent or significant disability or incapacity of the subject, is associated with a congenial anomaly or birth defect or is qualified as “other important medically significant event or condition”. These events were recorded by attending clinicians and entered into a database. Written informed consent was obtained from all patients participating in the study.

### Example 5—Forecasting of Continuous Vital Signs

As disclosed herein continuous monitoring of vital signs improved the foundation for data analysis with respect to standard care. The present example relates to prediction of vital signs. I.e. not only providing an alarm when deterioration has occurred, but actually predicting whether it is likely to occur in the near future.

The present example employs Multivariate Auto-Regressive (MAR) models to create a forecast projection of vital signs parameters based on past measurements. Forecasting vital signs could help identify deviation of the normal physiology that is likely to occur in the near future.

### Multivariate Auto-Regressive (MAR) Model

Consider a set of variables y=y₁, . . . , y_{N}, where each element yₜ=[yₜ₁, . . . , yₜₘ] is the response at time t, N is the signal length and m the number of modalities in the signal. The response at time, t, as defined by the MAR model is given by

$${y}_{t} = α + {\sum }_{k = 1}^{K} {β}_{k} \cdot {y}_{t - k} + ϵ$$

where α is a vector of m elements, βk is a matrix of size [m, m] from the array β=[βₖ, . . . , β_{K}]. Thus, in the auto regressive model the value of yₜ is given as a linear combination of the previous K elements of y, the intercept a and the weights in β.

Due to the nature of the vital signs signals, the physiological expectation of the temporal evolution in the signals is that homeostasis will cause the value to return to some patient specific baseline value. It can be advantageous to construct a model that includes the ‘pull’ towards a baseline value. This can be achieved by creating the MAR model centred around the intercept parameter. A popular implementation is to center the model around the mean of the signal, μ_{y}, where the response yₜ, computed in the equation above, instead comes from

$${y}_{t} = {μ}_{y} + {\sum }_{k = 1}^{K} {β}_{k} \cdot ( {y}_{t - k} - {μ}_{y} )$$

As the value of μ_{y}, when computed from the time series available, does not necessarily reflect the true baseline, this can be fixed globally or as a parameter fitted in the model.

### Data

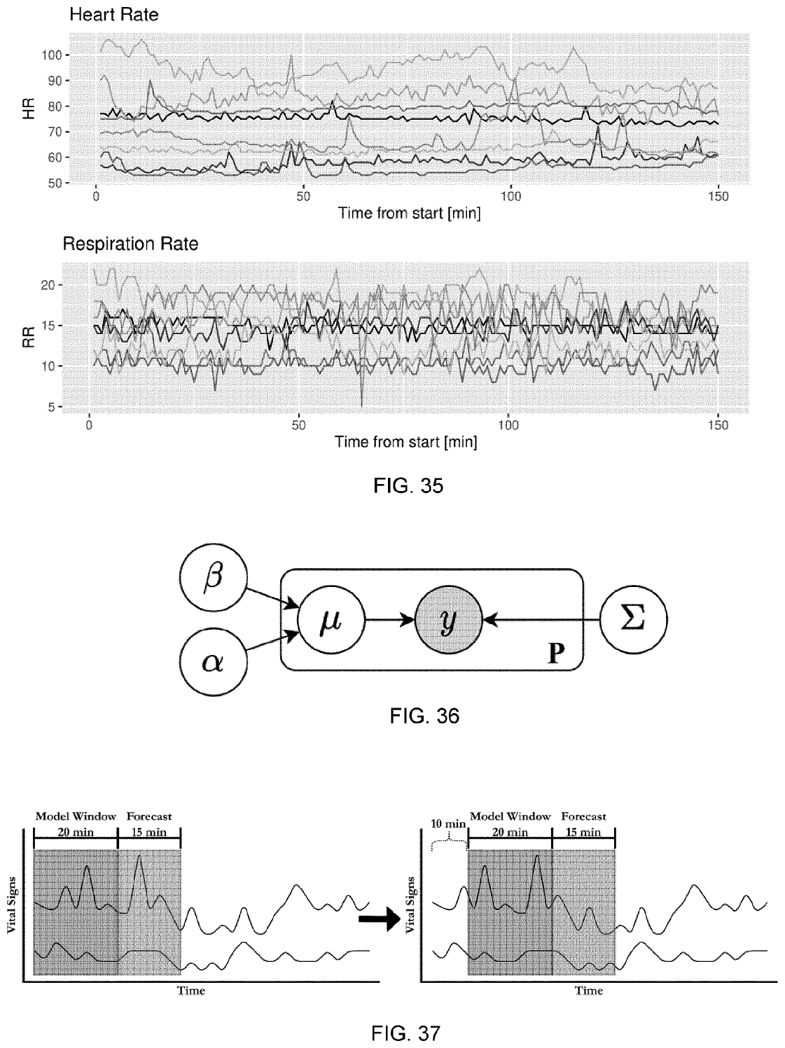

In this example data from an observational study with 500 postoperative cancer patients monitored for up to 4 days after major abdominal surgery was used. The data were obtained at Rigshospitalet and Bispebjerg Hospital in Copenhagen, Denmark from February 2018 to August 2020. Patients were monitored with a single lead ECG patch (Lifetouch Blue), a wrist-worn pulse oximeter (Nonin WristOx2), and a cuff-based blood pressure monitor (TM-2441). From the sensors the following modalities were available: Heart Rate (HR) (1/60 Hz), respiration rate (RR) (1/60 Hz), peripheral oxygen saturation (SpO2) (1/60 Hz) and systolic and diastolic blood pressure (measured every 30 minutes). All data were transmitted to a central server by the Isansys Patient Status Engine. A subset of the measurements were selected from the cohort, to perform the inference of the parameters in the model and evaluate the predictive accuracy. Only measurements of HR and RR values were used. The data extracted was ensured to not have any missing values for HR or RR in the time period. To fit the model, the subset consisted of 150 minutes of simultaneous HR and RR measurements from eight different patients chosen at random. This gave a total of 20 hours of data for inference. The time series used are shown in FIG. 35 showing time series vital signs for the patients used to fit the MAR model. To test the predictive accuracy of the model 400 minutes from five different patients were used. The patients in the subset used for evaluation were ensured to be different from the subset used for inference.

### Fitting the Model

The MAR model was constructed as a pooled model. A pooled model defines a model, where the same parameters are fitted across several different data sources, in this case different patients vital signs signals. This results in a single set of model parameters used for all future patients. In the case of the MAR model this means, that the parameters α, β and Σ are kept equal for all patients, P. The probabilistic graphical model of the implemented pooled MAR model is shown in the graph in FIG. 36.

For the model, the priors for the parameters were kept uninformative and given by normal distributions. As the intercept, α, is used as a global baseline, values for this were chosen to reflect common baseline values for the heart rate and respiration rate. For heart rate the mean was set to 70 and for respiration rate it was set to 12. All parameters in β had priors set to follow a standard normal distribution. The following summarizes the model

$$\begin{matrix}{y}_{t} \sim N ( {μ}_{t} , \sum ) \\ {μ}_{t} = α + {\sum }_{k = 1}^{K} ( {β}_{k} \cdot ( {y}_{t - k} - α ) )\end{matrix}$$

with the priors for α and β being

$$\begin{matrix}{α}_{HR} \sim N ( 70 , 10 ) \\ {α}_{RR} \sim N ( 12 , 4 ) \\ {β}_{*} \sim N ( 0 , 1 )\end{matrix}$$

The lag-parameter, K, was set to K=20, reflecting the past 20 minutes of vital signs data.

### Inference of Model Parameters

The objective for fitting a model is to establish the parameters of the model, θ, to fit the target distribution p(θ|y). This is done by either exact of approximate inference, depending on the dimensionality of the problem at hand. Due to the computational complexity of exact inference the current problem would be intractable in an exact approach. FIG. 37 illustrates the setup used for evaluating the model on new patients. For each step a forecast (right box) is performed based on the data available in the model window (left box). The windows are then moved 10 minutes forward and the process is repeated.

Instead approximate inference in the form of Markov Chain Monte Carlo (MCMC)-sampling is used. MCMC-sampling is a general method based on iteratively drawing samples of θ from approximate distributions and updating these to continuously improve the approximation of the target distribution. The idea is as in Bayesian simulation that the collection of the simulated draws from p(θ|y) will summarize the posterior density. Hence MCMC-sampling is useful for sampling from Bayesian posterior distributions, where it is intractable to infer θ exactly from p(θ|y). Due to the random initialization of the sampling algorithm, the samples will have a transition period from initialization to the posterior distribution. To account for this, a warm-up period is defined and the samples from this are rejected. To ensure that the sampling is stabilized at the posterior distribution, sampling from multiple independent chains were done such that convergence could be quantified by used of the diagnostic measure, Rb, which compares the within-chain variance and the between-chains variance. The idea is that while the individual chains have not mixed and thus not approached the target distribution, the variance of all chains mixed should be larger than that of the chains individually. As the individual chains converge, Rb-→1 and Vehtari et al. recommends R<b 1.01 before using the sample [10]. In the used setup, each model was fitted using 4 chains with 2000 iterations in each. Each chain was given a warmup period of 1000 iterations, thus leaving 1000 for sampling per chain. This provided 4000 posterior samples of the parameters.

### Evaluating the Predictive Accuracy

To evaluate the model's predictive accuracy, the model was applied to data from 5 unseen patients. A window matching the lag parameter, K=20, was provided to the model to create a forecast of 15 minutes. The forecast segment was compared to the true values within the window. For this, the root mean squared error (RMSE) was used to quantify the accuracy of the expected value in the forecast window with respect to the original signal. The window was moved 10 minutes forward and the process was repeated for the entirety of the time series. The setup for the first to steps is shown in FIG. 3

### Results

The results of the evaluation of the predictive accuracy of the forecasts are presented in the table below. The parameters, α, β and Σ, of the model showed proper convergence with all values of R<b 1.01. The average RMSE for HR across all patients was 11.4 bpm with the lowest and highest being 0.4 bpm and 32.1 bpm, respectively. For RR the average RMSE was 3.3 brpm with the lowest and highest being 0.9 brpm and 7.4 brpm, respectively. For HR the results in the table below show a large difference between patients, where the lowest average RMSE for one patient was 4.7 bpm and the highest 20.5 bpm. The resulting responses of the MAR model are visualized in FIG. 4. For visual purposes, the plots show the last 50 minutes leading up to the forecasting window and the 15 minutes within the forecasting window.

TABLE
RMSE between the forecast and the true values
RMSE - mean ± std

| Patient | Heart Rate [bpm] | Respiration Rate [brpm] |
|---|---|---|
| #1 | 20.5 ± 5.78 | 3.6 ± 1.29 |
| #2 | 4.7 ± 3.98 | 3.2 ± 1.37 |
| #3 | 6.7 ± 3.68 | 3.6 ± 1.45 |
| #4 | 14.0 ± 4.97 | 3.1 ± 1.38 |
| #5 | 11.1 ± 5.55 | 2.9 ± 0.94 |
| Average | 11.4 ± 7.30 | 3.3 ± 1.30 |

### Summary

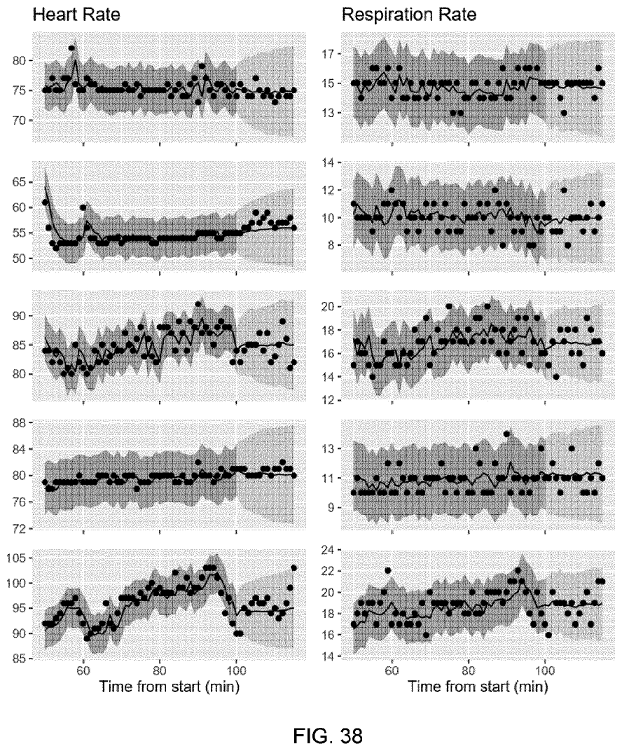

Predicting future deviations in vital signs, such as heart rate and respiration rate, is challenging, as the nature of the signals may imply rapid changes not known in advance. Sudden activation of the patients will lead to changes in their vital signs, that will not be possible to predict before the activation occur. The difficulty to capture this can be seen in FIG. 38, showing a visualization of the response of the hierarchical AR-model fitted to the HR and RR data. The legend “.” is the time series of the original signal, “-” is the expected value of the AR-model. (Grey area, to the left): Predictive interval (95%) of the AR-model. (Green area, to the right): Predictive interval (95%) of the forecasts from the AR-model, i.e. where rapid changes within the forecast window is not captured by the prediction.

The model proposed in this example demonstrates promising results when applied to different patients. The range in the subset used for evaluation shows that both in low and high values of HR and RR the model still provides a good forecast. Though, the variation occurring over multiple days and under different circumstances has only barely been assessed and there will most likely be rare events, that has not been represented in the evaluation. In this example, the model was implemented in a pooled construction which has advantages in a clinical setting. As the pooled model relies on a single set of parameters to span all patients, there is no requirement to perform inference of the parameters for each patient, which is resource demanding in computational power when done in an iterative Bayesian approach. This can also be a disadvantage of the pooled model compared to other constructions, such as the separate or hierarchical model, where patient specific variations can be built into the model. It could be advantageous if the model has difficulties in fitting to the diversity in data that different patients will present. However, as there is no clear patient specific deviation, use of these models must be held against the increased computational requirements.

Another aspect of the natural representation of vital signs, not included in this example, is the heteroscedasticity assumed to be present. The current model assumes the data to be homoscedastic within each modality, i.e. the data has the same variance across patients and temporal location. It becomes clear from the plots in FIG. 38, where plots one, two and four from above have very little variance in the data, and the third and fifth show a large variance, that the model assumption of homoscedasticity does not hold in reality. Two solutions to achieve heteroscedasticity could be to model the variance in 1) an auto-regressive manner likewise to the current modelling of the mean, or 2) to model the variance in a hierarchical way, where the parameters α, β and Σ relies on the variance in the data.

The construction of a model that creates a forecast will lead to the question of how to use the forecast. As the nature of the signals entails rapid changes, the conception that it will be possible to predict far into the future does not resemble reality. Instead, it could be advantageous to use the forecasts as baseline prediction and evaluate deviation from this based on the true values in a practical setup. Quantifying rapid changes from the forecast values could be a way to use the model to detect deviations in a real time setting.

The present example shows that it is possible to predict/forecast time series of the vital signs HR and RR based on previous measurements, for example by employing a pooled MAR model. Though there were large deviations in the predictive accuracy in the forecast window between patients, an fairly low RMSE of 11.4 bpm for HR and 3.3 bpm for RR was achieved on average, see for example FIG. 38. Hence, even though the work is based on a small subset of patient data, this example demonstrates promising results for forecasting vital signs in a clinical setting.

1. A computer-implemented method configured for automatic real-time detection of clinical deterioration events in a patient, the method comprising the steps of:
continuously receiving a plurality of different vital sign data from a plurality of sensors worn by the patient, the vital sign data comprising: electrocardiogram (ECG), photoplethysmogram (PPG), heart rate (HR), respiration rate (RR), blood pressure (e.g. systolic blood pressure, SBP), and peripheral oxygen saturation (SpO₂),
analyzing the vital sign data to identify artefacts;
discarding one or more data samples associated with the identified artefacts in the vital sign data in order to continuously obtain validated patient vital sign parameters;
executing a plurality of computer-implemented clinically validated deterioration event subroutines, each subroutine configured to receive one or more of the validated vital sign parameters and determine a specific clinical deterioration event in the patient, said deterioration event subroutines comprising:
bradypnea/apnea based on validated heart rate and respiration rate parameters,
tachypnea based on validated respiration rate,
hypoventilation based on validated respiration rate and peripheral arterial oxygen saturation,
desaturation based on validated peripheral arterial oxygen saturation,
sinus tachycardia based on validated heart rate, and
bradycardia based on validated heart rate,
hypotension based on validated systolic blood pressure or estimated systolic blood pressure,
hypertension based on validated systolic blood pressure or estimated systolic blood pressure,
providing an alarm when at least one of said deterioration events has been detected by one of said clinically validated automatic deterioration event subroutines.

2. The method according to claim 1, wherein the vital sign data further comprises heart rhythm and wherein the deterioration event subroutines further comprise asystole based on validated heart rhythm and pulse rate, and atrial fibrillation based on ECG and/or validated heart rhythm.

3. The method according to any of the preceding claims, wherein the deterioration event subroutines comprise: circulatory collapse based on validated heart rate and validated systolic blood pressure.

4. The method according to any of the preceding claims, wherein the vital sign data is selected from the group of: electrocardiogram (ECG), photoplethysmogram (PPG), heart rate (HR), respiration rate (RR), RR interval (RRI), blood pressure (e.g. systolic blood pressure, SBP), heart rhythm, ischemic ECG changes, peripheral temperature, peripheral skin conductance, 3D body position and acceleration, pulse rate, peripheral perfusion index, peripheral oxygen saturation (SpO₂), and subcutaneous glucose concentration.

5. The method according to any of the preceding claims, wherein the deterioration event subroutines is selected from the group of:
bradypnea/apnea based on validated heart rate and respiration rate parameters,
tachypnea based on validated respiration rate,
hypoventilation based on validated respiration rate and peripheral arterial oxygen saturation,
desaturation based on validated peripheral arterial oxygen saturation,
sinus tachycardia based on validated heart rate,
bradycardia based on validated heart rate,
hypotension based on validated systolic blood pressure,
circulatory collapse based on validated heart rate and systolic blood pressure,
asystole based on validated heart rhythm and pulse rate,
hypertension based on validated systolic blood pressure,
atrial fibrillation based on ECG and/or validated heart rhythm,
ventricular extrasystoles based on validated heart rhythm, and
ventricular tachycardia/-fibrillation based on validated ECG and/or validated heart rhythm,

6. The method according to any of the preceding claims, wherein the method further comprises one or more clinically validated deterioration event subroutines selected from the group of:
cardiac ischemia based on validated ischemic electrocardiographic response,
low perfusion index based on validated peripheral perfusion index, and
acute stress based on peripheral validated peripheral skin conductance and peripheral temperature.

7. The method according to any of the preceding claims, wherein the sensors are selected from the group of: electrocardiography (ECG) sensor, pulse oximeter, oscillometric blood pressure monitor, peripheral skin conductance sensor, 3D accelerometer, peripheral thermometer, and continuous glucose monitor.

8. The method according to any of the preceding claims, wherein the sensors are wireless sensors.

9. The method according to claim 7, wherein data from the sensors are streamed every minute or every 30 seconds.

10. The method according to any of the claims 7-9, wherein 10 seconds of ECG data are streamed every minute.

11. The method according to any of the claims 7-10, wherein the method further comprises an ECG preprocessing subroutine configured to assess the quality of the ECG data from an ECG sensor worn by the patient in order to obtain validated vital sign parameters such as validated HR, RR, heart rhythm and RRI parameters.

12. The method according to claim 11, wherein the ECG preprocessing subroutine comprises the steps of:
receiving ECG values from the ECG sensor, the ECG values comprising ECG time stamp, ECG samples, heart beat time stamp, R-R interval, and/or QRS amplitude;
discarding ECG values that are outside predefined thresholds, such as ECG<0 or ECG>4000 (the values referring to the amplitude of the R peak in the QRS complex), and/or discarding ECG values that are duplicate;
performing a linear interpolation of ECG missing values;
determining QRS index (i.e. the time associated with the location of the R peak in the QRS complex) in ECG using heart beat time stamp (hbTS);
comparing the number of heart beats and/or the QRS amplitude and/or the ratio between a sub selection of the QRS amplitudes and the number of heartbeats to one or more predefined thresholds, and in case said thresholds are exceeded, discarding one or more of the ECG values;
performing a bandpass filtering of the ECG values;
correcting R peak(s) in the QRS complexes from the ECG values and/or normalizing each heart beat from the ECG values;
calculating ecgTemplate (i.e. an average ECG heart cycle) or previously received ecgTemplate;
calculating correlation coefficients (cc);
comparing the average of cc and/or the number of cc to one or more predetermined thresholds, and in case said thresholds are exceeded, discarding one or more of the ECG values;
calculating the deviation between the corrected R peak(s) and the non-corrected R peak(s) and storing said deviation as HBdev value(s); and
comparing the HBdev value(s) to one or more predefined thresholds and discarding the ECG values that are associated with HBdev value(s) that exceed the one or more predefined thresholds.

13. The method according to any of the preceding claims, wherein the method further comprises an SpO₂ preprocessing subroutine configured to assess the quality of the SpO₂ values from a pulse oximeter worn by the patient in order to obtain validated SpO₂ values.

14. The method according to claim 13, wherein the SpO₂ preprocessing subroutine comprises the steps of:
receiving SpO₂ values from the pulse oximeter;
removing SpO₂ values that exceed one or more predefined thresholds such as SpO₂<0 and/or SpO₂>100;
removing duplicate SpO₂ values;
removing SpO₂ values that has a difference per second greater than 4;
extrapolating missing SpO₂ values; and
optionally calculating the average SpO₂ value from the SpO₂ values.

15. The method according to claim 14, wherein the one or more predefined thresholds comprise SpO_{2<0} and/or SpO₂>100.

16. The method according to any of the preceding claims, the method comprising a bradypnea subroutine configured to determine bradypnea/apnea, said subroutine comprising the steps of:
receiving validated HR and RR values;
comparing the validated HR and RR values to one or more predefined bradypnea thresholds, and
providing an alarm in case the HR and RR values exceed said predefined bradypnea thresholds for a predefined time duration.

17. The method according to claim 16, wherein the bradypnea thresholds comprise HR>20 bpm and RR≤5 bpm.

18. The method according to any of the claims 16-17, wherein the predefined time duration is ≥1 min.

19. The method according to any of the preceding claims, the method further comprising a tachypnea subroutine configured to determine tachypnea, said subroutine comprising the steps of:
receiving validated RR value(s);
comparing the validated RR value(s) to a predefined tachypnea threshold; and
providing an alarm in case the RR value(s) exceed the tachypnea threshold for a predetermined time duration.

20. The method according to claim 19, wherein the tachypnea threshold is RR≥24 bpm.

21. The method according to any of the claims 19-20, wherein the predefined time duration is ≥5 min.

22. The method according to any of the preceding claims, the method further comprising a hypoventilation subroutine configured to determine hypoventilation, said subroutine comprising the steps of:
receiving validated RR and SpO₂ value(s);
comparing each of the validated RR and SpO₂ values to one or more hypoventilation thresholds; and
providing an alarm in case in case the RR and SpO₂ values exceed the hypoventilation threshold(s) for a predefined time duration.

23. The method according to claim 22, wherein the hypoventilation thresholds comprise RR<11 bpm and SpO₂<88%.

24. The method according to any of the claims 22-23, wherein the predefined time duration is ≥5 min.

25. The method according to any of the preceding claims, the method further comprising a desaturation subroutine configured to determine desaturation, said subroutine comprising the steps of:
receiving validated SpO₂ values;
comparing the validated SpO₂ values to one or more predefined SpO₂ thresholds; and
providing an alarm in case the SpO₂ values exceed the SpO₂ threshold(s) for a predefined time duration t.

26. The method according to claim 25, wherein the predefined SpO₂ thresholds comprise any of: SpO₂<92%, or SpO₂<88%, or SpO₂<85%, or SpO₂<80%.

27. The method according to any of claims 25-26, wherein the desaturation subroutine provides an alarm in case:
$$\begin{matrix}{Sp O}_{2} < 92 \% for t \geq 60 min , or \\ {Sp O}_{2} < 88 \% for t \geq 10 min , or \\ {Sp O}_{2} < 85 \% for t \geq 5 min , or \\ {Sp O}_{2} < 80 \% for t \geq 1 min .\end{matrix}$$

28. The method according to any of the preceding claims, the method further comprising a sinus tachycardia subroutine configured to determine sinus tachycardia, said subroutine comprising the steps of:
receiving validated HR value(s);
comparing the validated HR values to one or more predefined sinus tachycardia thresholds; and
providing an alarm in case the HR values exceed the sinus tachycardia threshold(s) for a predefined time duration t.

29. The method according to claim 28, wherein the one or more predefined sinus tachycardia thresholds comprise HR≥111 bpm or HR>130 bpm.

30. The method according to any of the claims 28-29, wherein the sinus tachycardia subroutine provides an alarm in case: HR>130 bpm for t≥30 min, or in case HR≥111 bpm for t≥60 min.

31. The method according to any of the preceding claims, the method further comprising a bradycardia subroutine configured to determine bradycardia, said subroutine comprising the steps of:
receiving validated HR value(s);
comparing the validated HR values to one or more predefined bradycardia thresholds and/or ranges; and
providing an alarm in case the HR values exceed the bradycardia threshold(s) and/or ranges for a predefined time duration t.

32. The method according to claim 31, wherein the bradycardia threshold(s) comprise HR<30 bpm or 30 bpm≤ HR≤40 bpm.

33. The method according to any of the claims 31-32, wherein the bradycardia subroutine provides an alarm in case: HR<30 bpm for t≥1 min, or in case 30 bpm≤ HR≤40 bpm for t≥5 min.

34. The method according to any of the preceding claims, the method further comprising a hypotension subroutine configured to determine hypotension, said subroutine comprising the steps of:
receiving validated SBP value(s);
comparing the validated SBP values to one or more predefined hypotension thresholds; and
providing an alarm in case the SBP values exceed the hypotension threshold(s) for one or more consecutive measurements and/or for a predefined time duration t.

35. The method according to claim 34, wherein the hypotension thresholds comprise any of: SBP<70 mmHg and/or SBP<91 mmHg.

36. The method according to any of the claims 34-35, wherein the hypotension subroutine provides an alarm in case SBP<91 mmHg for two consecutive measurements, or in case SBP<70 mmHg.

37. The method according to any of the preceding claims, the method further comprising a circulatory collapse subroutine configured to determine circulatory collapse, said subroutine comprising the steps of:
receiving validated SBP and HR value(s);
comparing the validated SBP and HR values to one or more predefined SBP thresholds and HR thresholds; and
providing an alarm in case the SBP threshold(s) and at least one of the HR thresholds is exceeded for a predefined time duration t.

38. The method according to claim 37, wherein the predefined SBP threshold comprise SBP<100 mmHg and the predefined HR thresholds comprise any of HR>110 bpm, HR>130 bpm, and/or HR<50 bpm.

39. The method according to any of the claims 37-38, wherein the circulatory collapse subroutine provides an alarm in case:
$$\begin{matrix}S B P < 100 mm Hg and H R > 110 b p m for t \geq 30 min , or \\ S B P < 100 mm Hg and H R > 130 b p m for t \geq 5 min , or \\ S B P < 100 mm Hg and H R > 50 b p m for t \geq 30 min .\end{matrix}$$

40. The method according to any of the preceding claims, the method further comprising a asystole subroutine configured to determine asystole, said subroutine comprising the steps of:
receiving ECG data samples;
detecting QRS-complexes in the ECG data samples;
providing an alarm in case there are no QRS-complexes detected for a predefined time duration t1, and/or in case there is no peripheral pulse detected for a predefined time duration t2.

41. The method according to claim 40, wherein the predefined time durations t1 and t2 is selected from the list of: more than 10 seconds, more than 15 seconds, more than 20 seconds, more than 25 seconds, or more than 30 seconds.

42. The method according to any of the preceding claims, the method further comprising a hypertension subroutine configured to determine hypertension, said subroutine comprising the steps of:
receiving validated SBP value(s);
comparing the validated SBP values to one or more predefined hypertension thresholds; and
providing an alarm in case the SBP values exceed the hypertension threshold(s) for one or more consecutive measurements and/or for a predefined time duration t.

43. The method according to claim 42, wherein the hypertension thresholds comprise any of: SBP≥180 mmHg and/or SBP≥220 mmHg.

44. The method according to any of the claims 42-43, wherein the hypertension subroutine provides an alarm in case SBP≥180 mmHg for t≥60 min, or in case SBP≥220 mmHg.

45. The method according to any of the preceding claims, the method further comprising an atrial fibrillation subroutine configured to determine atrial fibrillation, said subroutine comprising the steps of:
receiving validated RRI values;
comparing the validated RRI values to one or more predefined first atrial fibrillation criterions, said criterion(s) specifying the validated RRI to be below or above one or more predefined threshold values;
optionally storing the validated values that fall within said first atrial fibrillation criterion in an array;
optionally comparing the amount of stored values in said array to a predefined second atrial fibrillation criterion relating to the size of said array; and
providing an alarm in case the first and/or second atrial fibrillation criterion is fulfilled.

46. The method according to claim 45, wherein the first atrial fibrillation criterion comprises RRI<200 or RRI>3000.

47. The method according to any of the claims 45-46, wherein the second atrial fibrillation criterion is that the size of the array is greater than 29.

48. The method according to any of the claims 45-47, wherein the atrial fibrillation subroutine further comprises the step of computing the normalized difference of the validated RRI values and storing the computed normalized difference values in a stored set of NDR values.

49. The method according to claim 48, wherein the atrial fibrillation subroutine further comprises the step of removing the NDR values that fall outside predefined percentiles of the values.

50. The method according to claim 49, wherein the predefined percentiles comprise the 10^{th} percentile and the 90^{th} percentile, such that NDR values that are below the 10^{th} percentile and/or above the 90^{th} percentile are discarded from the stored set of NDR values.

51. The method according to claim 50, wherein the atrial fibrillation subroutine further comprises the step of providing the stored set of NDR values to a Support Vector Machine (SVM) model configured for determining the presence of atrial fibrillation or absence of atrial fibrillation based on the NDR values.

52. The method according to claim 47, wherein an alarm is generated in case atrial fibrillation is present.

53. The method according to any of the preceding claims, further comprising the step of detecting and/or predicting one or more serious adverse events based on one or more of the determined clinical deterioration events.

54. A computer program having instructions which, when executed by a computing device or system, causes the computing device or system to execute the method according to any of the preceding claims, thereby providing automatic real-time detection of clinical deterioration events in a patient.

55. A system for automatic real-time detection of clinical deterioration events in a patient, comprising a non-transitive, computer-readable storage device for storing instructions that, when executed by a processor, performs the according to any of the preceding method claims.

56. A system for automatic detection of a clinical deterioration event in a patient, said system comprising:
one or more sensors configured for automatically monitoring vital sign data (such as heart rate, respiration rate, heart rate variability, temperature, oxygen saturation and blood pressure) of the patient, the one or more sensors further configured for transmitting the vital sign data wirelessly to a server and/or to a gateway;
a first server for receiving and storing the vital sign data, the first server having a computer program thereon, said computer program configured for executing the method according to any of the preceding method claims, thereby providing automatic detection of a clinical deterioration event in a patient; and
one or more gateways configured to provide a wireless communication link between the sensor(s) and the first server.
